SEQUENCE LISTING (11 sequences)

SEQ ID NO: 1 - DNA, 1185 bp, Artificial Sequence
  synthetic construct
atgaaaaaactcttgaaatcggtattagtgtttgccgctttgagttctgcttcctccttgcaagctctgcctgtggggaatcctgctgaaccaagccttatgatcgacggaattctatgggaaggtttcggcggagatccttgcgatccttgcaccacttggtgtgacgctatcagcatgcgtatgggttactatggtgactttgttttcgaccgtgttttgcaaacagatgtgaataaagaattccaaatgggtgccaagcctacaactgctacaggcaatgctgcagctccatccacttgtacagcaagagagaatcctgcttacggccgacatatgcaggatgctgagatgtttacaaatgctgcttacatggcattgaatatttgggatcgttttgatgtattctgtacattaggagccaccagtggatatcttaaaggaaattcagcatctttcaacttagttggcttattcggagataatgagaaccatgctacagtttcagatagtaagcttgtaccaaatatgagcttagatcaatctgttgttgagttgtatacagatactacttttgcttggagtgctggagctcgtgcagctttgtgggaatgtggatgcgcgactttaggcgcttctttccaatacgctcaatccaagcctaaagtcgaagaattaaacgttctctgtaacgcagctgagtttactatcaataagcctaaaggatatgtagggcaagaattccctcttgatcttaaagcaggaacagatggtgtgacaggaactaaggatgcctctattgattaccatgaatggcaagcaagtttagctctctcttacagactgaatatgttcactccctacattggagttaaatggtctcgagcaagttttgatgcagacacgattcgtattgctcagccgaagtcagctacaactgtctttgatgttaccactctgaacccaactattgctggagctggcgatgtgaaagctagcgcagagggtcagctcggagataccatgcaaatcgtttccttgcaattgaacaagatgaaatctagaaaatcttgcggtattgcagtaggaacaactattgtggatgcagacaaatacgcagttacagttgagactcgcttgatcgatgagagagctgctcacgtaaatgcacaattccgcttctaa

SEQ ID NO: 2 - protein, 394 aa, Artificial Sequence
  synthetic construct
MKKLLKSVLVFAALSSASSLQALPVGNPAEPSLMIDGILWEGFGGDPCDPCTTWCDAISMRMGYYGDFVFDRVLQTDVNKEFQMGAKPTTATGNAAAPSTCTARENPAYGRHMQDAEMFTNAAYMALNIWDRFDVFCTLGATSGYLKGNSASFNLVGLFGDNENHATVSDSKLVPNMSLDQSVVELYTDTTFAWSAGARAALWECGCATLGASFQYAQSKPKVEELNVLCNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSCGIAVGTTIVDADKYAVTVETRLIDERAAHVNAQFRF

SEQ ID NO: 3 - DNA, 1185 bp, Artificial Sequence
  synthetic construct
atgaaaaaactgctgaaatctgttctggttttcgcggcgctgtcttctgcgtcttctctgcaggcgctgccggttggtaacccggcggaaccgtctctgatgatcgacggtatcctgtgggaaggtttcggtggtgacccgtgcgacccgtgcaccacctggtgcgacgcgatctctatgcgtatgggttactacggtgacttcgttttcgaccgtgttctgcagaccgacgttaacaaagaattccagatgggtgcgaaaccgaccaccgcgaccggtaacgcggcggcgccgtctacctgcaccgcgcgtgaaaacccggcgtacggtcgtcacatgcaggacgcggaaatgttcaccaacgcggcgtacatggcgctgaacatctgggaccgtttcgacgttttctgcaccctgggtgcgacctctggttacctgaaaggtaactctgcgtctttcaacctggttggtctgttcggtgacaacgaaaaccacgcgaccgtttctgactctaaactggttccgaacatgtctctggaccagtctgttgttgaactgtacaccgacaccaccttcgcgtggtctgcgggtgcgcgtgcggcgctgtgggaatgcggttgcgcgaccctgggtgcgtctttccagtacgcgcagtctaaaccgaaagttgaagaactgaacgttctgtgcaacgcggcggaattcaccatcaacaaaccgaaaggttacgttggtcaggaattcccgctggacctgaaagcgggtaccgacggtgttaccggtaccaaagacgcgtctatcgactaccacgaatggcaggcgtctctggcgctgtcttaccgtctgaacatgttcaccccgtacatcggtgttaaatggtctcgtgcgtctttcgacgcggacaccatccgtatcgcgcagccgaaatctgcgaccaccgttttcgacgttaccaccctgaacccgaccatcgcgggtgcgggtgacgttaaagcgtctgcggaaggtcagctgggtgacaccatgcagatcgtttctctgcagctgaacaaaatgaaatctcgtaaatcttgcggtatcgcggttggtaccaccatcgttgacgcggacaaatacgcggttaccgttgaaacccgtctgatcgacgaacgtgcggcgcacgttaacgcgcagttccgtttctaa

SEQ ID NO: 4 - DNA, 1119 bp, Artificial Sequence
  synthetic construct
ctgccggttggtaacccggcggaaccgtctctgatgatcgacggtatcctgtgggaaggtttcggtggtgacccgtgcgacccgtgcaccacctggtgcgacgcgatctctatgcgtatgggttactacggtgacttcgttttcgaccgtgttctgcagaccgacgttaacaaagaattccagatgggtgcgaaaccgaccaccgcgaccggtaacgcggcggcgccgtctacctgcaccgcgcgtgaaaacccggcgtacggtcgtcacatgcaggacgcggaaatgttcaccaacgcggcgtacatggcgctgaacatctgggaccgtttcgacgttttctgcaccctgggtgcgacctctggttacctgaaaggtaactctgcgtctttcaacctggttggtctgttcggtgacaacgaaaaccacgcgaccgtttctgactctaaactggttccgaacatgtctctggaccagtctgttgttgaactgtacaccgacaccaccttcgcgtggtctgcgggtgcgcgtgcggcgctgtgggaatgcggttgcgcgaccctgggtgcgtctttccagtacgcgcagtctaaaccgaaagttgaagaactgaacgttctgtgcaacgcggcggaattcaccatcaacaaaccgaaaggttacgttggtcaggaattcccgctggacctgaaagcgggtaccgacggtgttaccggtaccaaagacgcgtctatcgactaccacgaatggcaggcgtctctggcgctgtcttaccgtctgaacatgttcaccccgtacatcggtgttaaatggtctcgtgcgtctttcgacgcggacaccatccgtatcgcgcagccgaaatctgcgaccaccgttttcgacgttaccaccctgaacccgaccatcgcgggtgcgggtgacgttaaagcgtctgcggaaggtcagctgggtgacaccatgcagatcgtttctctgcagctgaacaaaatgaaatctcgtaaatcttgcggtatcgcggttggtaccaccatcgttgacgcggacaaatacgcggttaccgttgaaacccgtctgatcgacgaacgtgcggcgcacgttaacgcgcagttccgtttctaa

SEQ ID NO: 5 - DNA, 63 bp, Artificial Sequence
  synthetic construct
atgaaaaagacagctatcgcgattgcagtggcactggctggtttcgctaccgtagcgcaggcc

SEQ ID NO: 6 - protein, 21 aa, Artificial Sequence
  synthetic construct
MKKTAIAIAVALAGFATVAQA

SEQ ID NO: 7 - DNA, 1182 bp, Artificial Sequence
  synthetic construct
atgaaaaagacagctatcgcgattgcagtggcactggctggtttcgctaccgtagcgcaggccctgccggttggtaacccggcggaaccgtctctgatgatcgacggtatcctgtgggaaggtttcggtggtgacccgtgcgacccgtgcaccacctggtgcgacgcgatctctatgcgtatgggttactacggtgacttcgttttcgaccgtgttctgcagaccgacgttaacaaagaattccagatgggtgcgaaaccgaccaccgcgaccggtaacgcggcggcgccgtctacctgcaccgcgcgtgaaaacccggcgtacggtcgtcacatgcaggacgcggaaatgttcaccaacgcggcgtacatggcgctgaacatctgggaccgtttcgacgttttctgcaccctgggtgcgacctctggttacctgaaaggtaactctgcgtctttcaacctggttggtctgttcggtgacaacgaaaaccacgcgaccgtttctgactctaaactggttccgaacatgtctctggaccagtctgttgttgaactgtacaccgacaccaccttcgcgtggtctgcgggtgcgcgtgcggcgctgtgggaatgcggttgcgcgaccctgggtgcgtctttccagtacgcgcagtctaaaccgaaagttgaagaactgaacgttctgtgcaacgcggcggaattcaccatcaacaaaccgaaaggttacgttggtcaggaattcccgctggacctgaaagcgggtaccgacggtgttaccggtaccaaagacgcgtctatcgactaccacgaatggcaggcgtctctggcgctgtcttaccgtctgaacatgttcaccccgtacatcggtgttaaatggtctcgtgcgtctttcgacgcggacaccatccgtatcgcgcagccgaaatctgcgaccaccgttttcgacgttaccaccctgaacccgaccatcgcgggtgcgggtgacgttaaagcgtctgcggaaggtcagctgggtgacaccatgcagatcgtttctctgcagctgaacaaaatgaaatctcgtaaatcttgcggtatcgcggttggtaccaccatcgttgacgcggacaaatacgcggttaccgttgaaacccgtctgatcgacgaacgtgcggcgcacgttaacgcgcagttccgtttctaa

SEQ ID NO: 8 - protein, 393 aa, Artificial Sequence
  synthetic construct
MKKTAIAIAVALAGFATVAQALPVGNPAEPSLMIDGILWEGFGGDPCDPCTTWCDAISMRMGYYGDFVFDRVLQTDVNKEFQMGAKPTTATGNAAAPSTCTARENPAYGRHMQDAEMFTNAAYMALNIWDRFDVFCTLGATSGYLKGNSASFNLVGLFGDNENHATVSDSKLVPNMSLDQSVVELYTDTTFAWSAGARAALWECGCATLGASFQYAQSKPKVEELNVLCNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSCGIAVGTTIVDADKYAVTVETRLIDERAAHVNAQFRF

SEQ ID NO: 9 - DNA, 1182 bp, Artificial Sequence
  synthetic construct
atgaaaaagacagctatcgcgattgcagtggcactggctggtttcgctaccgtagcgcaggccctgccggttggtaacccggcggaaccgtctctgatgatcgacggtatcctgtgggaaggtttcggtggtgacccgtctgacccgtctaccacctggtctgacgcgatctctatgcgtatgggttactacggtgacttcgttttcgaccgtgttctgcagaccgacgttaacaaagaattccagatgggtgcgaaaccgaccaccgcgaccggtaacgcggcggcgccgtctacctctaccgcgcgtgaaaacccggcgtacggtcgtcacatgcaggacgcggaaatgttcaccaacgcggcgtacatggcgctgaacatctgggaccgtttcgacgttttctctaccctgggtgcgacctctggttacctgaaaggtaactctgcgtctttcaacctggttggtctgttcggtgacaacgaaaaccacgcgaccgtttctgactctaaactggttccgaacatgtctctggaccagtctgttgttgaactgtacaccgacaccaccttcgcgtggtctgcgggtgcgcgtgcggcgctgtgggaatctggttctgcgaccctgggtgcgtctttccagtacgcgcagtctaaaccgaaagttgaagaactgaacgttctgtctaacgcggcggaattcaccatcaacaaaccgaaaggttacgttggtcaggaattcccgctggacctgaaagcgggtaccgacggtgttaccggtaccaaagacgcgtctatcgactaccacgaatggcaggcgtctctggcgctgtcttaccgtctgaacatgttcaccccgtacatcggtgttaaatggtctcgtgcgtctttcgacgcggacaccatccgtatcgcgcagccgaaatctgcgaccaccgttttcgacgttaccaccctgaacccgaccatcgcgggtgcgggtgacgttaaagcgtctgcggaaggtcagctgggtgacaccatgcagatcgtttctctgcagctgaacaaaatgaaatctcgtaaatcttctggtatcgcggttggtaccaccatcgttgacgcggacaaatacgcggttaccgttgaaacccgtctgatcgacgaacgtgcggcgcacgttaacgcgcagttccgtttctaa

SEQ ID NO: 10 - protein, 393 aa, Artificial Sequence
  synthetic construct
MKKTAIAIAVALAGFATVAQALPVGNPAEPSLMIDGILWEGFGGDPSDPSTTWSDAISMRMGYYGDFVFDRVLQTDVNKEFQMGAKPTTATGNAAAPSTSTARENPAYGRHMQDAEMFTNAAYMALNIWDRFDVFSTLGATSGYLKGNSASFNLVGLFGDNENHATVSDSKLVPNMSLDQSVVELYTDTTFAWSAGARAALWESGSATLGASFQYAQSKPKVEELNVLSNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSSGIAVGTTIVDADKYAVTVETRLIDERAAHVNAQFRF

SEQ ID NO: 11 - protein, 372 aa, Artificial Sequence
  synthetic construct
LPVGNPAEPSLMIDGILWEGFGGDPSDPSTTWSDAISMRMGYYGDFVFDRVLQTDVNKEFQMGAKPTTATGNAAAPSTSTARENPAYGRHMQDAEMFTNAAYMALNIWDRFDVFSTLGATSGYLKGNSASFNLVGLFGDNENHATVSDSKLVPNMSLDQSVVELYTDTTFAWSAGARAALWESGSATLGASFQYAQSKPKVEELNVLSNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSSGIAVGTTIVDADKYAVTVETRLIDERAAHVNAQFRF

(19) United States
(12) Patent Application Publication
(10) Pub. No.: US 2021/0106668 A1
(43) Pub. Date: Apr. 15, 2021

(54) RECOMBINANT CHLAMYDIA-ACTIVATED B CELL PLATFORMS AND METHODS OF USE THEREOF

(71) Applicant: Ohio State Innovation Foundation, Columbus, OH (US)

(72) Inventors: Thomas CHERPES, Stanford, CA (US); Rodolfo D. VICETTI MIGUEL, Palo Alto, CA (US); Natividad RUIZ, Columbus, OH (US)

(21) Appl. No.: 16/496,686

(22) PCT Filed: Mar. 23, 2018
(86) PCT No.: PCT/US2018/024007

§ 371 Date: Sep. 23, 2019

Related U.S. Application Data
(60) Provisional application No. 62/475,413, filed on Mar. 23, 2017.

(51) Int. Cl. A61K 39/118; C07K 14/295

(52) U.S. Cl. CPC A61K 39/118; A61K 2039/575; C07K 14/295

(57) ABSTRACT

Disclosed herein is a recombinant Chlamydia-activated B cell (CAB) platform, vaccines, and methods of using the same. Disclosed is a method of enhancing a population of B cells, comprising exposing said B cells to a recombinant polypeptide derived from a Chlamydia spp. under conditions suitable to enhance the population of B cells, such that expansion and differentiation of said B cells takes place, and said B cells are exposed or crosslinked to an antigen. Also disclosed are methods to use a recombinant polypeptide derived from a Chlamydia spp. as an adjuvant for induction of enhanced humoral immunity against an unrelated antigen (e.g., humoral immunity against HIV antigen). Also disclosed are methods of producing said CABs, and treating a subject in need thereof with said CABs.

### CROSS-REFERENCE TO RELATED APPLICATIONS

This application claims benefit of U.S. Provisional Application No. 62/475,413, filed Mar. 23, 2017, incorporated herein by reference in its entirety.

### BACKGROUND

Dendritic cells (DCs) are considered potent antigen-presenting cells (APCs), and effective inducers of protective immunity against infectious diseases and cancers. These properties have prompted intense interest in the use of DCs as cellular vaccines; especially DCs differentiated from peripheral blood monocytes. However, clinical trials using DCs have only demonstrated very low rates of overall clinical response, highlighting the need to improve DC-based vaccines. Particular restrictions for the success of these cellular therapies have been the limited number of DCs that can be produced from monocytes, as DC cannot be expanded ex vivo, making it difficult to generate large numbers of these cells for use in long-term, multi-administration protocol. Moreover, DCs have a significant degree of variability in their ability to prime immune responses after cryopreservation. These limitations become especially important because greater DC numbers and treatments have been shown to elicit more robust antitumor immunity and improve clinical responses.

B cells represent a large pool of potent APCs, and are likely the only autologous APC alternative to DC that can be generated ex vivo for immunotherapeutic purposes. While B cells have been described to induce T cell tolerance or even to block antitumor immune responses in vivo, these reports were restricted to resting B cells lacking important expression of accessory and costimulatory molecules. B cells activated to become effective APCs by cells expressing CD40L in combination with cytokines or Toll-like receptor (TLR) ligands likewise did not induce optimal B cell activation or required the use of cell lines. These limitations made them unsuitable for clinical application.

Activated B cells have enhanced MHC and costimulatory molecule expression, and exhibit greatly improved antigen presentation capacity to fully activate naïve and memory T cells. Also of importance, activated B cells recruit T cells through the secretion of chemokines and migrate to secondary lymphoid organs; critical requirements for in vivo induction of effective antitumor immune responses. Because B cells are easily obtained ex vivo, they are an attractive source of autologous APCs for immunotherapeutic applications. Moreover, activated B cells express MHC class I and II molecules and thus can be used with a wide range of antigens. Hence, a practical method that increases activation and proliferation of B cells is needed in the art to provide a cellular vaccine to target multiple types of tumors and infectious diseases.

### SUMMARY

Disclosed herein is a cellular vaccine platform that uses Chlamydia-activated B cells as immunotherapy, as well as methods of using the platform. Disclosed herein are recombinant versions of Chlamydia trachomatis major outer membrane protein (MOMP) that elicit robust B cell activation. These recombinant proteins can be used to generate Chlamydia-activated B cells (CABs), which can be used as immunogens in Chlamydia-based vaccines. These recombinant proteins can also be used to increase the production and affinity of antibodies against other unrelated antigens.

Specifically, disclosed herein is a platform for creating activated, antigen-presenting cells (APCs), wherein the platform comprises: a polypeptide or glycopolypeptide comprising a derivative of naturally occurring major outer membrane protein (MOMP) of a Chlamydia spp., wherein the naturally occurring MOMP is modified so that at least one cysteine residue of MOMP is replaced with a different amino acid; a population of B cells; and an antigen, wherein the antigen is not derived from a Chlamydia spp. Also disclosed is a vaccine created using the platform described herein. Further described is a Chlamydia-activated B cell produced by the platform described herein.

Disclosed herein is a method for producing activated, antigen-presenting Chlamydia-activated B cells from a subject, the method comprising: transforming Escherichia coli with a plasmid, wherein the plasmid comprises a nucleic acid encoding major outer membrane (MOMP) protein of Chlamydia and a wild-type or altered signal sequence of E. coli OmpA or other E. coli protein, wherein E. coli then produces recombinant MOMP; exposing B cells of the subject to the recombinant MOMP; and exposing the B cells to a desired antigen, wherein the antigen is not derived from Chlamydia spp., thereby obtaining activated, antigen-presenting Chlamydia-activated B cells (CABs).

Disclosed herein is a method of treating a subject in need thereof, the method comprising: obtaining B cells from the subject; exposing the B cells to a polypeptide comprising a derivative of naturally occurring major outer membrane protein (MOMP) of a Chlamydia spp., wherein the naturally occurring MOMP is modified so that at least one cysteine residue of MOMP is replaced with a different amino acid; exposing the B cells to an antigen, wherein the antigen is not derived from Chlamydia spp., thereby obtaining activated, antigen-presenting Chlamydia-activated B cells (CABs); and treating the subject with the activated, antigen-presenting Chlamydia-activated B cells.

Further disclosed is a vaccine comprising an antigen, wherein the antigen is not derived from a Chlamydia spp, and a polypeptide comprising a derivative of naturally occurring major outer membrane protein (MOMP) of a Chlamydia spp., wherein the naturally occurring MOMP is modified so that at least one cysteine residue of MOMP is replaced with a different amino acid.

The details of one or more embodiments of the invention are set forth in the accompa-nying drawings and the description below. Other features, objects, and advantages of the invention will be apparent from the description and drawings, and from the claims.

### DESCRIPTION OF DRAWINGS

FIG. 1 shows a schematic representation of the generation of E. coli K-12 strain expressing recombinant C. trachomatis MOMP. Construction of engineered E. coli strains that display MOMP on their outer membrane is accomplished by expressing the plasmid-encoded C. trachomatis ompA (ompA_{Ct}) in E. coli K-12.

FIG. 2 shows a diagram indicating processing and assembling that recombinant C. trachomatis MOMP undergoes in E. coli K-12. Outer membrane proteins (OMPs) must be transported, processed, and assembled into β-barrels. The signal sequence (SS) targets the pro-protein to the Sec translocon, which translocates the protein across the inner membrane (IM). The signal peptidase cleaves the SS and the mature protein is targeted and assembled into a β-barrel conformation with the help of periplasmic chaperones and the β-barrel assembly machine (Bam complex).

FIG. 3 shows a schematic demonstrating how E. coli OmpA signal sequence (SSEc) use allows adequate transport and folding of recombinant C. trachomatis MOMP (Ct-MOMP) in the outer membrane.

FIG. 4A-E shows MOMP production in E. coli strains carrying the pET23/42ChOmpA plasmid. Production of MOMP can be induced in strain NR4424 [genotype KRX (pET23/42ChOmpA)] (Panel A and B) with the addition of L-rhamnose (Panel C). Growth in terrific broth (TB) for 3 h or overnight (0/N) at either room temperature (−20° C., RT) or 30° C. results in the production of recombinant Ct-MOMP. Ct-MOMP can be detected in gels stained with Coomassie Blue (Panel D) or anti-MOMP antiserum (Panel E). Band corresponding to Ct-MOMP is marked with arrow. Samples from control strain NR4432 [genotype KRX (pET23/42)], which carries the vector control lacking the Ct-MOMP-encoding gene, were also tested.

FIG. 5A-B shows ability of C. trachomatis MOMP overexpressed in E. coli to induce proliferation of human B cells in vitro when combined with a TLR1/2 agonist. Fluorescently labeled B cells and CD4⁺ T cells, isolated by negative immunomagnetic selection from human PBMCs, were exposed to inactivated E. coli transfected with vector KRX (pET23/42) (vector only) or KRX (pET23/42ChOmpA) (MOMP), or were left unstimulated. Fluorescently labeled B cells and CD4⁺ T cells were also exposed to identical conditions in the presence of Pam3CSK4 (a TLR1/2 agonist), as indicated. Flow cytometric analysis of proliferating B cells was performed after 4 days. (A) Representative dot plots show proliferation of human B cells in response to E. coli overexpressing MOMP. (B) Fold increase in normalized B cell proliferation for cells exposed to both strains of inactivated E. coli in the presence of TLR1/2 agonist.

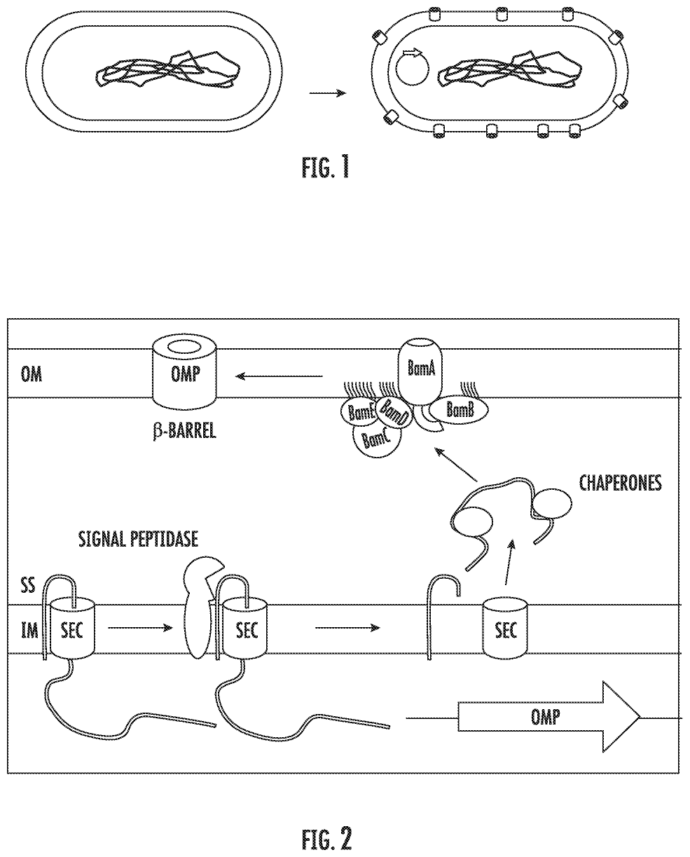

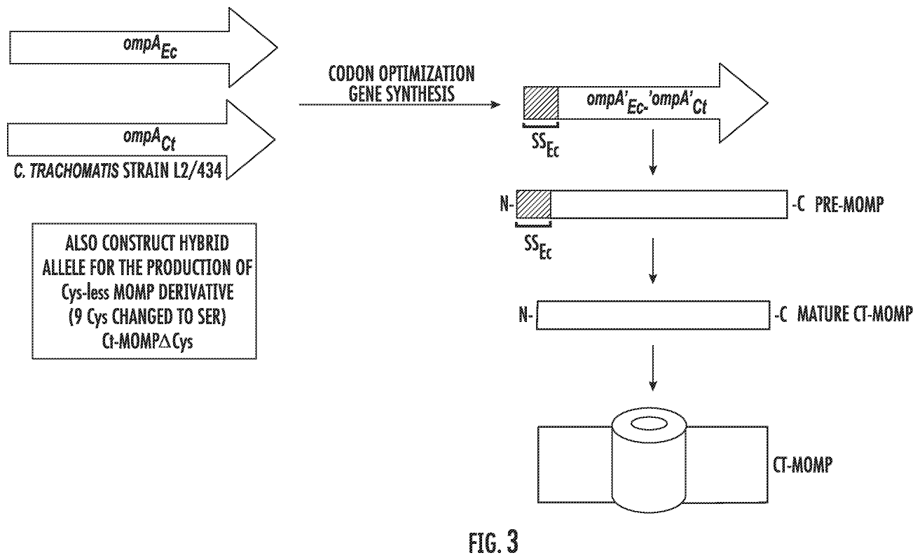

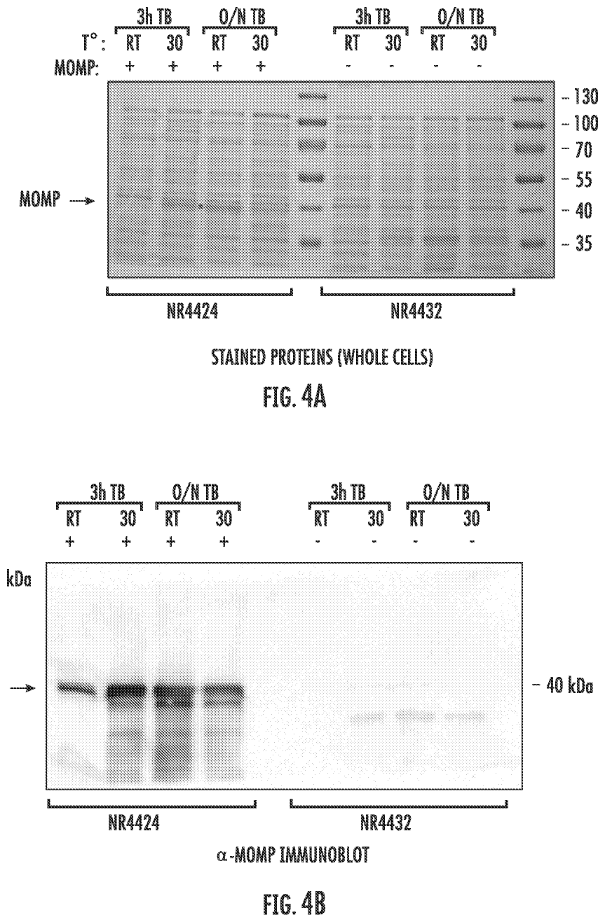

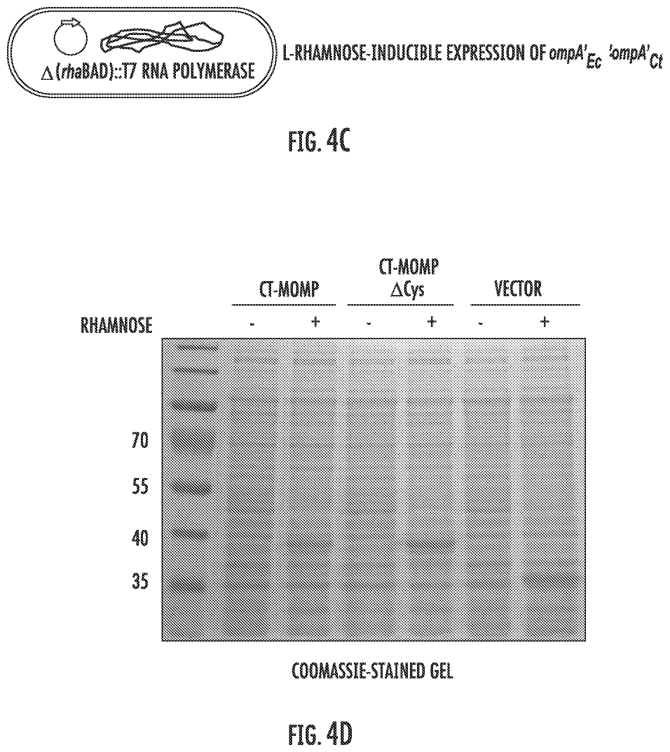

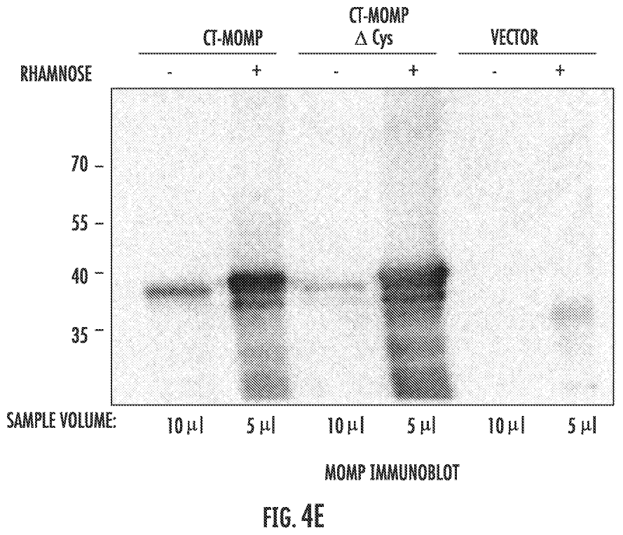

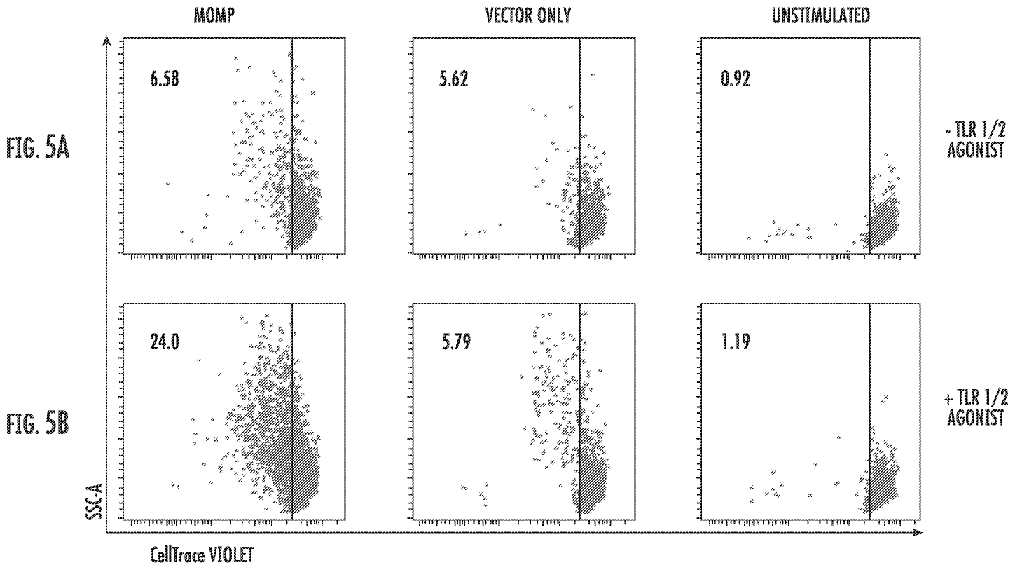

### DETAILED DESCRIPTION

### Definitions

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of .+−0.20% or .+−0.10%, more preferably .+−0.5%, even more preferably .+−0.1%, and still more preferably .+−0.0.1% from the specified value, as such variations are appropriate to perform the disclosed methods.

The term “antigenic composition” refers to a composition comprising material which stimulates the immune system and elicits an immune response in a host or subject.

The term “elicit an immune response” refers to the stimulation of immune cells in vivo in response to a stimulus, such as an antigen. The immune response consists of both cellular immune responses, e.g., T cell and macrophage stimulation, and humoral immune responses, e.g., B cell and complement stimulation and antibody production. Immune response may be measured using techniques well-known in the art, including, but not limited to, antibody immunoassays, proliferation assays, and others.

A “prophylactic” treatment is a treatment administered to a subject who does not exhibit signs of a disease or exhibits only early signs for the purpose of decreasing the risk of developing pathology. The vaccines disclosed herein can be given as a prophylactic treatment to reduce the likelihood of developing a pathology or to minimize the severity of the pathology, if developed.

A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective.

The term “inactivated” is used herein to describe a microorganism, such as Chlamydia spp. (including C. trachomatis, C. psittaci and C. muridarum), that is also known in the art as a “killed” or “dead” microorganism. An inactivated bacterium is a whole bacterium without infective properties and is produced from a “live” bacterium, regardless of whether the bacterium has been previously attenuated in any manner.

A “fragment” of a polypeptide refers to any portion of the polypeptide smaller than the full-length polypeptide or protein expression product. Fragments are, in one aspect, deletion analogs of the full-length polypeptide wherein one or more amino acid residues have been removed from the full-length polypeptide. Accordingly, “fragments” are a subset of deletion analogs described below.

The term “antibody,” as used herein, refers to an immunoglobulin molecule which can bind a specific epitope on an antigen, often with a high degree of specificity and affinity. Antibodies can be intact immunoglobulins derived from natural sources or from recombinant sources and can be immunoreactive portions of intact immunoglobulins. Antibodies can be produced from the vaccines described herein, and may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, to “alleviate” a disease means to reduce the frequency or severity of at least one sign or symptom of a disease or disorder.

An “effective amount” as used herein, means an amount which provides a therapeutic or prophylactic benefit.

As used herein, an “immunoassay” refers to any binding assay that uses an antibody capable of binding specifically to a target molecule to detect and quantify the target molecule.

As used herein, an “instructional material” includes a publication, a recording, a diagram, or any other medium of expression which can be used to communicate the usefulness of a compound, composition, method, platform, or system of the invention in the kit for practicing the methods described herein. The instructional material of the kit of the invention can, for example, be affixed to a container which contains the identified compound, composition, platform, or delivery system of the invention or be shipped together with a container which contains the identified compound, composition, method components, platform, or system of the invention. Alternatively, the instructional material can be shipped separately from the container with the intention that the instructional material and the compound be used cooperatively by the recipient.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Also included are glycopeptides, which are peptides that contain carbohydrate moieties (glycans) covalently attached to the side chains of the amino acid residues that constitute the peptide.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side effects. The effect of therapy will also be impacted by the physiological state of a subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity, and the age, weight, etc., of the subject to be treated.

To “treat” a disease as the term is used herein, means to reduce the frequency or severity of at least one sign or symptom of a disease or disorder experienced by a subject.

The term “cell” as used herein refers to individual cells, cell lines, primary culture, or cultures derived from such cells unless specifically indicated. A “culture” refers to a composition comprising isolated cells of the same or a different type. A cell line is a culture of a particular type of cell that can be reproduced indefinitely, thus making the cell line “immortal.” A cell culture can be a population of cells grown on a medium such as agar. A primary cell culture is a culture from a cell or taken directly from a living organism, which is not immortalized.

The term “biological sample” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The terms “tumor cell” or “cancer cell”, used either in the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. The term “tumor-associated antigen” or “TAA” is used herein to refer to a molecule or complex which is expressed at a higher frequency or density by tumor cells than by non-tumor cells of the same tissue type. Tumor-associated antigens may be antigens not normally expressed by the host; they may be mutated, truncated, misfolded, or otherwise abnormal manifestations of molecules normally expressed by the host; they may be identical to molecules normally expressed but expressed at abnormally high levels; or they may be expressed in a context or milieu that is abnormal. Tumor-associated antigens may be, for example, proteins or protein fragments, complex carbohydrates, gangliosides, haptens, nucleic acids, or any combination of these or other biological molecules. Knowledge of the existence or characteristics of a particular tumor-associated antigen is not necessary for the practice of the invention.

The term “B cell” refers to a B lymphocyte. B cell precursors reside in the bone marrow where immature B cells are produced. B cell development occurs through several stages, each stage representing a change in the genome content at the antibody loci. In the genomic heavy chain variable region there are three segments, V, D, and J, which recombine randomly, in a process called VDJ rearrangement to produce a unique variable region in the immunoglobulin of each B cell. Similar rearrangements occur for the light chain variable region except that there are only two segments involved, V and J. After complete rearrangement, the B cell reaches the IgM⁺ immature stage in the bone marrow. These immature B cells present a membrane bound IgM, i.e., BCR, on their surface and migrate to the spleen, where they are called transitional B cells. Some of these cells differentiate into mature B lymphocytes. Mature B cells expressing the BCR on their surface circulate the blood and lymphatic system performing the role of immune surveillance. They do not produce soluble antibodies until they become fully activated. Each B cell has a unique receptor protein that will bind to one particular antigen. Once a B cell encounters its antigen and receives an additional signal from a T helper cell, it can further differentiate into a plasma B cell expressing and secreting soluble antibodies or a memory B cell.

The term “B cell” can also refer to any B lymphocyte which presents a fully rearranged, i.e., a mature, B cell receptor (BCR) on its surface. For example, a B cell can be an immature or a mature B cell and is preferably a naïve B cell, i.e., a B cell that has not been exposed to the antigen specifically recognized by the BCR on the surface of said B cell. The B cells can be memory B cells, preferably IgG⁺ memory B cells. The term “B cells” can also refer to a mixture of B cells. A mixture of B cells can mean that the B cells in the mixture have different antigen-specificities, i.e., produce antibodies or fully rearranged BCRs which recognize a variety of antigens. The antibodies or BCRs of a single B cell are usually identical, also with respect to antigen-specificity.

The term “B cell secreting antibodies” preferably refers to plasma B cells. The term “B cells carrying a BCR on their surface” preferably refers to B cells expressing a BCR, preferably a fully rearranged BCR, at their plasma membrane. In this context, “a BCR” preferably does not mean a single BCR but preferably means a multitude of BCRs having the same antigen.

The term “portion” refers to a fraction. A portion preferably means at least 20%, at least 30%, preferably at least 40%, preferably at least 50%, more preferably at least 60%, more preferably at least 70%, even more preferably at least 80%, and most preferably at least 90% of the entire entity. The term “substantial portion” preferably refers to at least 50%, more preferably at least 60%, more preferably at least 70%, even more preferably at least 80%, even more preferably at least 90%, even more preferably at least 95%, and most preferably at least 99% of the entire entity.

The term “clonal expansion” refers to a process wherein a specific entity is multiplied. In context of the present invention, the term is preferably used in the context of an immunological response in which lymphocytes, preferably B lymphocytes, are stimulated by an antigen, proliferate, and the specific lymphocyte recognizing said antigen is amplified. Preferably, clonal expansion leads to differentiation of the lymphocytes, preferably into lymphocytes producing and secreting antibodies. B lymphocytes secreting antibodies are, for example, plasma B cells.

The term “antigen” relates to an agent comprising an epitope against which an immune response is to be generated. The term “antigen” includes, in particular, proteins, peptides, polysaccharides, lipids, nucleic acids, especially RNA and DNA, and nucleotides. The term “antigen” also includes derivatized antigens as secondary substance which becomes antigenic—and sensitizing—only through transformation (e.g., intermediately in the molecule, by completion with body protein), and conjugated antigens which, through artificial incorporation of atomic groups (e.g., isocyanates, diazonium salts), display a new constitutive specificity. In a preferred embodiment, the antigen is a tumor antigen, i.e., a constituent of cancer cells which may be derived from the cytoplasm, the cell surface and the cell nucleus, in particular those antigens which are produced, preferably in large quantity, intracellularly or as surface antigens on tumor cells. Examples are carcinoembryonic antigen, a 1-fetoprotein, isoferritin and fetal sulfoglycoprotein, a2-H-ferroprotein and γ-fetoprotein and various viral tumor antigens. In a further embodiment, the antigen is a viral antigen such as viral ribonucleoproteins or envelope proteins. In particular, the antigen or peptides thereof should be recognizable by a B cell receptor or an immunoglobulin molecule such as an antibody. Preferably, the antigen, if recognized by a B cell receptor, is able to induce in presence of appropriate co-stimulatory signals, polyclonal/clonal expansion of the B cell carrying the BCR specifically recognizing the antigen, and the differentiation of such B cells into antibody secreting B cells. An antigen can present in a repetitive organization, i.e., the antigen comprises more than one, preferably at least 2, at least 3, at least 4, up to 6, 10, 12 or more agents or epitopes against which an immune response is to be generated or against which the antibodies which are to be produced. Such repetitive antigen preferably is capable of binding to more than one antibody of the same specificity. In other words, such repetitive antigen comprises more than one epitope, preferably identical epitope, and thus is capable of “crosslinking” antibodies directed to said epitope. The more than one agents or epitopes may be covalently or non-covalently linked, wherein a covalent linkage may be by any chemical grouping such as by peptide linkages. An antigen can be a fusion molecule comprising a repetition of an antigen peptide or comprising different antigen peptides having a common epitope. In one preferred embodiment, said antigen peptides are linked by peptide linkers.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

### General

Disclosed herein is a cellular vaccine platform that uses Chlamydia-activated B cells as immunotherapy, as well as methods of using the platform. Disclosed herein are recombinant versions of Chlamydia trachomatis major outer membrane protein (MOMP) that elicit robust B cell activation. These recombinant proteins can be used to generate Chlamydia-activated B cells (CABs). Incorporated by reference herein are platforms, vaccines, and methods of using CABs, such as those found in PCT Application WO/2016/1320667.

Disclosed herein is a humoral vaccine that uses recombinant versions of Chlamydia trachomatis major outer membrane protein (MOMP) in combination with one or more antigens, which are not related to Chlamydia, to increase the production and affinity of antibodies against these antigens.

Specifically, Escherichia coli strains have been engineered to produce derivatives of MOMP. In one example, the native MOMP is derived from C. trachomatis strain L2/434 (SEQ ID NOS: 1 and 2 disclose native MOMP nucleic acid and amino acid sequence, respectively). However, it is noted that any chlamydial outer membrane proteins can be used in accordance with the methods described herein. For example, a MOMP derivative can be provided wherein the MOMP is native, but wherein it further comprises a wild-type or altered signal sequence from E. coli. Nucleic acid encoding an E. coli signal sequence can be found in SEQ ID NO: 5, and its corresponding polypeptide (or glycopolypeptide) can be found in SEQ ID NO: 6. These signal sequences can be used with any MOMP or MOMP derivative. Examples of nucleic acids encoding MOMP fused to an E. coli signal sequence can be found in SEQ ID NOS: 7 and 9, with corresponding amino acid sequences found in SEQ ID NOS: 8 and 10.

Also disclosed herein are MOMPs wherein one or more cysteine residues are replaced with an alternate amino acid. For example, 1, 2, 3, 4, 5, 6, 7, 8, 9, or more cysteine residues can be replaced with another amino acid. In one example, all cysteine residues can be replaced with another amino acid. In one example, one or more of the cysteine residues can be replaced with serine. Replacing cysteine residues can improve expression of MOMP.

The MOMP derivatives disclosed herein can be assembled in the outer membrane of an engineered E. coli strain, for example. Properly folded recombinant MOMPs can display the ability to activate mammalian B cells, can serve as immunogens in Chlamydia vaccines, and can serve as an adjuvant in humoral vaccines.

In one example, the E. coli strains carry pET23/42 derived plasmids encoding the MOMP derivatives. These derivatives are encoded by recombinant genes designed to optimize the transport and assembly of MOMP at the outer membrane. To optimize targeting of MOMP to the inner membrane Sec translocon and processing into the mature form, the 5′ end of these genes can encode the signal sequence of the E. coli ompA gene. This 5′ end can be fused in-frame to the codon-optimized portion of the Chlamydial ompA gene encoding mature wild-type or cysteine-less MOMP, as described herein.

Either whole MOMP proteins, or fragments thereof, can be used with the methods and platforms described herein. One of skill in the art can readily identify fragments of chlamydial proteins that are responsible for activation of mammalian B cells, as well as the precise epitopes needed for a CAB-based vaccine, for example. The methods and platforms described herein can be used with such fragments. In addition, whole E. coli cells expressing MOMP derivatives and purified recombinant MOMP proteins can be used with or without further processing for the application described above.

The nucleic acids disclosed herein can further comprise a purification tag. For example, the addition of a His6-tag or His8-tag can be used to allow purification of fusion proteins (i.e., chimera). In order to purify folded proteins from the outer membranes of a host E. coli strain, for example, tags can be added at the junction between the signal sequence of OmpA_{Ec} and mature MOMP_{Ct} sequence (Cys-containing and Cys-less versions). The resulting mature MOMP derivatives can contain tags at their N-terminus, for example. In order to purify unfolded proteins from the cytoplasm (inclusion bodies) of host E. coli strains, the sequence encoding the signal sequence can be replaced with a start codon followed by the tag. The unfolded, mature MOMP derivatives will contain tags at their N-terminus and can be refolded after purification using standard, published methods.

Also disclosed herein are antigenic fragments of MOMP. For example, disclosed herein are several methods and platforms that comprise the use of MOMP or an antigenic fragment thereof. One of skill in the art will appreciate that the entire sequence of MOMP need not be used with the methods and platforms disclosed herein, but rather any portion of MOMP that exhibits properties which allow for its use with the methods and platforms can be used. For example, U.S. Patent Application No. US 2014/0275478 A1 (herein incorporated by reference in its entirety) discloses surface-exposed fragments of Chlamydia trachomatis (Ct), and repeats thereof, for maximal antibody response against itself or unrelated antigen (if used as an adjuvant). These surface-exposed fragments can be extended to cover the flanking region of the surface-exposed fragments that may contain T cell epitopes. One example is a defined large fragment representing an extended version of the VD1 or VD4 region from the Ct MOMP antigen and in the immuno-repeat format provides high levels of surface binding and neutralizing antibodies against Ct. These surface-exposed repeats can be recombinantly fused with fragments of other surface exposed antigens such as PMPs or OMPs. The active fragments can also be used for the induction of antibodies against antigens unrelated to Chlamydia.

Regarding the sequences disclosed herein, “identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Also disclosed herein are changes in MOMP to identify domains responsible for B-cell activation: site-directed mutagenesis of plasmid encoding MOMP_{Ct} variants (Cys-containing, Cys-less, tagged versions, for example) can be used to alter the sequence of mature MOMP_{Ct} derivatives in order to identify domains responsible for B cell activation.

Therefore, disclosed herein is a platform for creating activated, antigen-presenting cells (APCs), wherein the platform comprises: a polypeptide or glycopolypeptide comprising a derivative of naturally occurring major outer membrane protein (MOMP) of a Chlamydia spp., wherein the naturally occurring MOMP is modified so that at least one cysteine residue of MOMP is replaced with a different amino acid; a population of B cells; and an antigen, wherein the antigen is not derived from a Chlamydia spp. Also disclosed is a vaccine created using the platform described herein. Further described is a Chlamydia-activated B cell produced by the platform described herein.

Disclosed herein is a method for producing activated, antigen-presenting Chlamydia-activated B cells from a subject, the method comprising: transforming Escherichia coli with a plasmid, wherein the plasmid comprises a nucleic acid encoding major outer membrane (MOMP) protein of Chlamydia and a wild-type or altered signal sequence of E. coli OmpA or other E. coli protein, wherein E. coli then produces recombinant MOMP; exposing B cells of the subject to the recombinant MOMP; and exposing the B cells to a desired antigen, wherein the antigen is not derived from Chlamydia spp., thereby obtaining activated, antigen-presenting Chlamydia-activated B cells (CABs).

Further disclosed in the method above, is a step wherein after exposing the B cells to a polypeptide comprising a derivative of major outer membrane protein (MOMP) of a Chlamydia spp., wherein the naturally occurring MOMP is modified so that at least one cysteine residue of MOMP is replaced with a different amino acid; adding the step of crosslinking a protein, peptide, nucleic acid, lipid, carbohydrate or fragment thereof to the CABs, wherein the protein, peptide, nucleic acid, lipid, carbohydrate or fragment thereof is antigenic.

Disclosed herein is a method of treating a subject in need thereof, the method comprising: obtaining B cells from the subject; exposing the B cells to a polypeptide comprising a derivative of naturally occurring major outer membrane protein (MOMP) of a Chlamydia spp., wherein the naturally occurring MOMP is modified so that at least one cysteine residue of MOMP is replaced with a different amino acid; exposing the B cells to an antigen, wherein the antigen is not derived from Chlamydia spp., thereby obtaining activated, antigen-presenting Chlamydia-activated B cells (CABs); and treating the subject with the activated, antigen-presenting Chlamydia-activated B cells.

This method further comprises the step of wherein after exposing the B cells to a polypeptide comprising a derivative of naturally occurring major outer membrane protein (MOMP) of a Chlamydia spp., wherein the naturally occurring MOMP is modified so that at least one cysteine residue of MOMP is replaced with a different amino acid; adding the step of crosslinking a protein, peptide, nucleic acid, lipid, carbohydrate or fragment thereof to the CABs, wherein the protein, peptide, nucleic acid, lipid, carbohydrate or fragment thereof is an antigen.

Further disclosed is a vaccine comprising an antigen and a polypeptide comprising a derivative of naturally occurring major outer membrane protein (MOMP) of a Chlamydia spp. or a fragment thereof, wherein the naturally occurring MOMP is modified so that at least one cysteine residue of MOMP is replaced with a different amino acid.

The antigen and a protein, peptide, or carbohydrate of Chlamydia spp., or whole inactivated Chlamydia or a fragment thereof, can be chemically conjugated to the antigen for use as an adjuvant. Alternatively, the antigen and a protein, peptide, or carbohydrate of Chlamydia spp., or whole inactivated Chlamydia or a fragment thereof, can simply be combined without conjugation. Nanoparticles can also be used, wherein the nanoparticle is coated with a protein, peptide, or carbohydrate of Chlamydia spp., or whole inactivated Chlamydia. The protein, peptide, or carbohydrate of Chlamydia spp., or whole inactivated Chlamydia spp., can be conjugated to antigen or just combined with antigen (not chemically bound) prior to administration via the nanoparticle.

By stating that the “antigen is not derived from a Chlamydia spp.” is meant that the antigen is not a protein, peptide, carbohydrate, nucleic acid, or fragment of any of these, which was obtained from Chlamydia spp. In other words, the antigen is derived from a source other than Chlamydia, such as another infectious agent, or from a tumor. For example, the antigen can share less than 90, 80, 70, 60, 50, 40, 30, 20, or 10% homology with a protein or nucleic acid of Chlamydia.

The protein, peptide, or carbohydrate of Chlamydia spp. (or fragment thereof) can be any functional fragment which is capable of eliciting the desired immune response. The Chlamydia spp. (including C. trachomatis, C. psittaci and C. muridarum) used in the vaccine and methods disclosed herein can be live, inactivated, or can be a protein, carbohydrate, or a fragment from Chlamydia spp. (including C. trachomatis, C. psittaci and C. muridarum). The Chlamydia spp. can be a variant of the known species, and still retain the function of imparting the effect disclosed herein. For example, the entire bacteria can be used (live bacteria do not infect leukocytes and cannot survive in an antibiotic-containing culture medium). Alternatively, inactivated whole bacteria (X-ray or gamma-irradiated) or lysate generated from the whole bacteria can be used. In another embodiment, specific proteins, carbohydrates, or fragments thereof can be used. For example, Chlamydia trachomatis major outer membrane protein (MOMP) or a fragment thereof can be used. In another example, inactivated C. trachomatis elementary bodies (EB) or reticulate bodies (RB) can be used. One of skill in the art can readily determine which protein, peptide, or carbohydrate of Chlamydia spp., or fragment thereof, can be used to impart the desired effect.

Any antigen from any disease, disorder, or condition may be used. Exemplary antigens include but are not limited to bacterial, viral, parasitic, allergens, autoantigens and tumor-associated antigens. If a DNA based vaccine is used, the antigen will typically be encoded by a sequence of the administered DNA construct. Alternatively, if the antigen is administered as a conjugate, the antigen will typically be a protein comprised in the administered conjugate. Particularly, the antigen can include protein antigens, peptides, whole inactivated organisms, and the like.

A single antigen can be used with the vaccines and methods disclosed herein, or multiple antigens can be used together. Examples include 2, 3, 4, or more antigens used in the same vaccine, or administered concurrently, or within a certain time frame of each other.

In one aspect, the antigen is selected from or derived from the group consisting of rotavirus, foot and mouth disease virus, influenza A virus, influenza B virus, influenza C virus, H1N1, H2N2, H3N2, H5N1, H7N7, H1N2, H9N2, H7N2, H7N3, H1 0N7, human parainfluenza type 2, herpes simplex virus. Epstein Barr virus, tularemia, Variola major (smallpox), viral hemorrhagic fevers, Yersinia pestis (plague), varicella virus, porcine herpesvirus 1, Listeria, cytomegalovirus, Lyssavirus, Poliovirus, Hepatitis A, Hepatitis B, Hepatitis C, Hepatitis D, Hepatitis E, distemper virus, Venezuelan equine encephalomyelitis, feline leukemia virus, reovirus, respiratory syncytial virus, Lassa fever virus, polyoma tumor virus, canine parvovirus, papilloma virus, tick borne encephalitis virus, Rinderpest virus, human rhinovirus species, Enterovirus speciees, Mengo virus, paramyxovirus, avian infectious bronchitis virus, human T-cell leukemia-lymphoma virus 1, human immunodeficiency virus-1, human immunodeficiency virus-2, lymphocytic choriomeningitis virus, paro-virus B 19, adenovirus, rubella virus, yellow fever virus, dengue virus, bovine respiratory syncytial virus, Corona virus, Bordetella pertussis, Bordetella bronchiseptica, Bordetella parapertussis, Brucella aborlis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, Mycobacterium tuberculosis, Mycobacterium avium, Mycobacterium bovis (Bacille Calmette Guerin), Mycobacterium leprae, Pneumococci, Staphylococci, Enterobacter species, Rochalimaua henselae, Pasterurella haemolytica, Pasterurella multocida, Treponema pallidum, Haemophilus species, Mycoplasma bovigenitalium, Mycoplasma pulmonis, Mycoplasma species, Borrelia burgdorferi, Legionalla pneumophila, Colstridium botutlinum, Corynebacterium diphtheriae, Yersinia entercolitica, Rickettsia rickettsii, Rickettsia typhi, Rickettsia prowsaekii, Ehrlichia chaffeensis, Anaplasma phagocytophilum, Plasmodium falciparum, Plasmodiun vivax, Plasmodium malariae, Schistosomes, Trypanosomes, Leishmania species, Filarial nematodes, Trichomoniasis, Sarcosporidiasis, Taenia saginata, Taenia solium, Leishmania, Toxoplasma gondii, Trichinella spiralis, Coccidiosis, Eimeria tenella, Cryptococcus neoformans, Candida albican, Apergillus fumigatus, Coccidioidomycosis, Neisseria gonorrhoeae, Malaria circumsporozoite protein, Malaria merozoite protein, Trypanosome surface antigen protein, Pertussis, Alphaviruses, Adenovirus, Diphtheria toxoid, Tetanus toxoid, meningococcal outer membrane protein, Streptococcal M protein, Influenza hemagglutinin, cancer antigen, tumor antigens, toxins, exotoxins, Neurotoxins, cytokines, cytokine receptors, monokines, monokine receptors, plant pollens, animal dander, and dust mites. Other antigens include antigens associated with autoimmune conditions, inflammatory conditions, allergy, asthma, and transplant rejection.

Specifically, disclosed herein is a vaccine strategy for administering HIV-1 envelope glycoproteins (Env) and Chlamydia, which promotes both accelerated affinity maturation of HIV-specific antibody and development of antibodies with HIV-specific neutralizing activity.

Much effort has been put towards that development of Env that have the immunogenic characteristics necessary to elicit effective antibody responses with broad HIV-1 neutralizing activity. However, due to conformational and glycan shielding of conserved Ab determinants on the virus spike, HIV-1 is a highly neutralization-resistant virus. Eliciting broadly neutralizing antibodies that bind poorly to more accessible epitope regions on Env is therefore extremely challenging and requires selective targeting of specific sub-determinants and the use of potent adjuvants that increase and/or accelerate affinity maturation. As indicated above, Chlamydia has substantial capacity to work as an adjuvant that increases affinity maturation to antigens associated with HIV-1 or other microbial pathogens.

Disclosed is a kit comprising an antigen and a polypeptide comprising a derivative of naturally occurring major outer membrane protein (MOMP) of a Chlamydia spp. or an antigenic fragment thereof, wherein the naturally occurring MOMP is modified so that at least one cysteine residue of MOMP is replaced with a different amino acid, wherein the antigen is not derived from a Chlamydia spp.

Also disclosed is a method of preventing disease or infection in a subject, the method comprising administering to the subject an antigen, wherein the antigen is a derivative of naturally occurring major outer membrane protein (MOMP) of a Chlamydia spp., wherein the naturally occurring MOMP is modified so that at least one cysteine residue of MOMP is replaced with a different amino acid; thereby preventing a disease or infection in the subject.

Using this platform, animal (e.g., mouse, cat, dogs and rhesus macaques) and human B cells obtained from peripheral blood or secondary lymphoid organs can be activated and expanded in vitro for infusion into a recipient using a variety of administration protocols. The B cells can be obtained from and used in the same individual (autologous), or the B cells can be obtained from one individual and used in another individual (allogenic). These cells can be activated in vitro by culture of peripheral blood mononuclear cells or whole lymphoid organ cell preparations in the presence of the recombinant MOMP peptides disclosed herein, inducing their activation and proliferation, which are further enhanced by additional factors, such as cytokines. The number of activated B cells can be expanded many fold from the initial number of B cells. For example, the number of activated B cells can be expanded by 2, 3, 4, 5, 6, 7, 8, 9, 10, 11, 12, 13, 14, 15, 16, 17, 18, 19, or 20-fold or more when compared to a control. It is shown herein that these cells are efficient APCs, capable of processing foreign protein antigens, presenting immunogenic peptides and stimulating allogeneic, naïve CD4⁺ and CD8⁺ T cells as well as naïve and memory antigen-specific CD8⁺ T cells. Additionally, antigens can be loaded by crosslinking to CABs to increase their therapeutic capacity.

Expanding the number of efficient APCs available for loading with cognate antigens makes the process of producing autologous cellular vaccines more effective, since in vivo administration of these activated B cells primes robust CD8⁺ T cell responses, capable of rejecting tumors and controlling viral infection. Therefore, a cellular vaccine platform has been developed for use in immunotherapy against tumors and infectious diseases. This platform and associated method is less invasive, costly, and labor intensive that other currently available cellular vaccine options.

CABs produced under the conditions disclosed herein can be combined with any desired antigen or combination of antigens, as well as with immunogenic peptides, by a variety of techniques known to those of skill in the art. CABs can be administered intravenously to the subject, for example. The magnitude of T cell proliferation and activation is dependent on the number of APCs administered. Due to the high number of cells that can be obtained with the methods disclosed herein, T cell responses induced by repeated administration of high numbers of CABs is greater than the ones induced by currently available preparation of DCs, because of their limited numbers. For example, the CABs disclosed herein can be pulsed with cognate tumor antigens or tumor-specific peptides and induce tumor-specific CD8⁺ T cells responses, capable of rejecting the corresponding tumor in murine tumor challenge models.

As a result of the disclosed method of activating B cells, CABs can be used in a wide range of approaches to present a desired antigen, such as a tumor-associated antigen to T cells. Human CABs are very efficient APCs and stimulate proliferation of human allogeneic naïve CD4⁺ and CD8⁺ T cells. They can also prime autologous naïve and memory T cells specific for viral and tumor antigens in humans and other mammals. Furthermore, in mice, these T cell responses are capable of rescuing from lethal viral infections and regressing established tumors.

The original B cells used herein can be obtained from a number of sources, including peripheral blood mononuclear cells, bone marrow, lymph node tissue, cord blood, tissue from a site of infection, splenic tissue, and tumors. Any number of B cell lines available in the art can be used with the platforms and methods disclosed herein. In certain embodiments of the methods described herein, B cells can be obtained from a unit of blood collected from a subject using any number of techniques known to the skilled artisan, such as FICOLL™ (copolymers of sucrose and epichlorohydrin that may be used to prepare high density solutions) separation.

MOMP can be derived from any Chlamydia spp. (including C. trachomatis, C. psittaci, C. pneumoniae and C. muridarum, for example). In addition to MOMP, other proteins can also be used to enhance CAB formation. For example, Chlamydia trachomatis polymorphic membrane proteins (e.g. PmpA, PmpB, PmpC, PmpD, PmpE, PmpF, PmpG, PmpH, PmpI); Chlamydia protein associating with death domains (CADD); Chlamydial protease-like activity factor (CPAF); Major outer membrane protein and cysteine-rich proteins (e.g. OmcA and OmcB); Chlamydia trachomatis 70-kDa heat shock protein; PulD/YscC; PorB; CTL0887; CTL0541; OprB; OMP85; CTL0645; Pal; Ef-Tu/TufA; GroEL; CopD; DnaK/HSP70; CTL0255; Hc1; CTL0850; and RpoB. Further disclosed are Chlamydiae components as found in Heinz et al. (Comprehensive in silico Prediction and Analysis of Chlamydial Outer Membrane Proteins Reflects Evolution and Lifestyle of the Chlamydiae. BMC Genomics. 2009 Dec. 29; 10:634) can be used.

Immunogenic antigens from a vast number of diseases, disorders, or conditions may be used with the platform described herein. Exemplary antigens include but are not limited to bacterial, viral, parasitic, allergens, autoantigens and tumor-associated antigens. If a DNA based vaccine is used, the antigen will typically be encoded by a sequence of the administered DNA construct. Alternatively, if the antigen is administered as a conjugate, the antigen will typically be a protein comprised in the administered conjugate. Particularly, the antigen can include protein antigens, peptides, whole inactivated organisms, and the like.

Specific examples of antigens that can be used include, but are not limited to, antigens from hepatitis A, B, C or D, influenza virus, Listeria, Clostridium botulinum, tuberculosis, tularemia, Variola major (smallpox), viral hemorrhagic fevers, Yersinia pestis (plague), HIV, herpes, papilloma virus, and other antigens associated with infectious agents. Other antigens include antigens associated with autoimmune conditions, inflammatory conditions, allergy, asthma, and transplant rejection. An antigen-loaded CAB can be administered alone or in conjunction with other therapeutic agents, such as a CD40 agonist or TLR ligands, in particular, an anti-CD40 agonist antibody, for use as a therapeutic or prophylactic vaccine for treating a disease condition. In another example, the CAB platform disclosed herein can be used in conjunction with checkpoint inhibitors. Examples of checkpoint inhibitor technology can be found in WO1999015157A2, WO2015016718A1, and WO2010149394A1, which are hereby incorporated in their entireties for their disclosure concerning checkpoint inhibitors. Other combination therapies are discussed herein as well.

In one embodiment, the antigen can comprise a tumor-related antigen. Examples of tumors that can be treated include the following: pancreatic tumors, such as pancreatic ductal adenocarcinoma; lung tumors, such as small and large cell adenocarcinoma, squamous cell carcinoma, and bronchoalveolar carcinoma; colon tumors, such as epithelial adenocarcinoma and their metastases; and liver tumors, such as hepatoma and cholangiocarcinoma. Also included are breast tumors, such as ductal and lobular adenocarcinoma; gynecologic tumors, such as squamous and adenocarcinoma of the uterine cervix, and uterine and ovarian epithelial adenocarcinoma; prostate tumors, such as prostatic adenocarcinoma; bladder tumors, such as transitional squamous cell carcinoma; tumors of the RES system, such as nodular or diffuse B or T cell lymphoma, plasmacytoma, and acute or chronic leukemia; skin tumors, such as malignant melanoma; and soft tissue tumors, such as soft tissue sarcoma and leiomyosarcoma. Of especial interest are brain tumors, such as astrocytoma, oligodendroglioma, ependymoma, medulloblastomas, and primitive neural ectodermal tumor. Included in this category are gliomas, glioblastomas, and gliosarcomas.

Specifically, the following antigens are associated with the following types of cancer, and can be used in the platforms and methods disclosed in Table 1:

TABLE 1
Cancers and Associated Antigens

| | |
|---|---|
| Melanoma | Tyrosinase, Tyrosinase-related protein (Trp- |
| | 1), gp100, Melan/MART-1 |
| Prostate adenocarcinoma | Prostate-specific membrane antigen, Prostate- |
| | specific acid phosphatase, Prostate specific |
| | antigen |
| Pancreatic, lung, breast and colon | MUC1 |
| adenocarcinoma | |
| Non-small-cell lung carcinoma | MUC1, MAGE antigens, EGFR |
| Cancer/testis antigens | LAGE/NY-ESO1, MAGE antigens, CEA, |
| | AFP |
| Breast cancer | HER-2 |
| Acute myelogenous leukemia | Aurora-A kinase, BRAP, Cyclin A1, hTert, |
| | WT1 |
| Chronic lymphocytic leukemia | ROR1 |
| Chronic myelogenous leukemia | BCR/ABL, BRAP, CML28, CML66, PR1, |
| | Proteinase 3, survivin, WT1 |

The immune status of the individual may be any of the following: The individual may be immunologically naïve with respect to certain tumor-associated antigens present in the composition, in which case the compositions may be given to initiate or promote the maturation of an anti-tumor response. The individual may not currently be expressing anti-tumor immunity, but may have immunological memory, particularly T cell memory relating to a tumor-associated antigen comprised in the vaccine, in which case the compositions may be given to stimulate a memory response. The individual may also have active immunity (either humoral or cellular immunity, or both) to a tumor-associated antigen comprised in the vaccine, in which case the compositions may be given to maintain, boost, or maturate the response, or recruit other arms of the immune system. The subject should be at least partly immunocompetent, so that the vaccine can induce endogenous T cell responses.

In another embodiment, the antigen can comprise an infectious agent. Examples of infectious agents which can be treated using the platforms and methods disclosed herein include, but are not limited to, Influenza viruses, Respiratory Syncytial Virus (RSV), Human Papilloma Virus (HPV), Hepatitis B Virus (HBV), Hepatitis C Virus (HCV), Human T-Lymphotrophic Virus Type-1, Human Immunodeficiency Virus 1 (HIV-1), Epstein-Barr Virus (EBV), Cytomegalovirus and other Herpesviridae. Other examples include Listeria monocytogenes, Salmonella, Mycobacterium tuberculosis, Plasmodium sp. (Malaria), Toxoplasma gondii, and Trypanosoma cruzi. Specifically, it has been shown that CABs can be used to immunize mice and protect them against ocular infection with HSV-2 (Herpesviridae), which in mice is a lethal infection.

The CABs disclosed herein can be exposed or crosslinked to more than one antigen simultaneously, or sequentially. For example, the CABs disclosed herein can be exposed to 2, 3, 4, 5, 6, or more antigens simultaneously or sequentially, or CABs loaded with different single antigens can be combined together for administration.

The CABs disclosed herein can be significantly expanded as compared to a population of B cells not exposed to Chlamydia spp. (including C. trachomatis, C. psittaci, C. pneumoniae and C. muridarum) or a protein or fragment thereof. As used herein expansion of B cells includes stimulation of proliferation of the cells as well as prevention of apoptosis or other death of the cells. As used herein, “culturing” and “incubation” are used to indicate that the cells are maintained in cell culture medium for a period of time with the appropriate additives (feeder cells, cytokines, agonists, other stimulatory molecules or media, which may include buffers, salts, sugars, serum or various other constituents). Those of skill in the art will appreciate that the culturing or incubation time may be varied to allow proper expansion, adjust for different cell densities or frequencies of individual subsets, and allow an investigator to properly time use of the cells. Thus the precise culture length may be determined empirically by one of skill in the art.

The CABs can have increased major histocompatibility complex (MHC) and and/or costimulatory molecule expression levels compared to inactive or resting B cells. For example, the CABs can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 90, 100% higher MHC and/or costimulatory molecule expression level compared to inactive B cells, or to B cells which have not been exposed to Chlamydia spp. (including C. trachomatis, C. psittaci, C. pneumoniae and C. muridarum) or a protein or fragment thereof. The CABs can have 2, 3, 4, 5, 6, 7, 8, 9, 10, 11, 12, 13, 14, 15, 16, 17, 18, 19, 20, or more fold higher MHC and costimulatory molecule expression level compared to inactive B cells, or to B cells which have not been exposed to Chlamydia spp. (including C. trachomatis, C. psittaci, C. pneumoniae and C. muridarum) or a protein or fragment thereof.

The CABs can have improved capacity to present antigen and activate T cells as compared to inactive B cells. By “improved capacity” is meant that they have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 90, or 100% more capacity to present antigen and activate T cells compared to a population of cells which have not been exposed to Chlamydia spp. (including C. trachomatis, C. psittaci, C. pneumoniae and C. muridarum) or a protein or fragment thereof. The CABs can have 2, 3, 4, 5, 6, 7, 8, 9, 10, 11, 12, 13, 14, 15, 16, 17, 18, 19, 20, or more fold capacity to present antigen and activate T cells compared to a population of cells which have not been exposed to Chlamydia spp. (including C. trachomatis, C. psittaci, C. pneumoniae and C. muridarum) or a protein or fragment thereof.

The CABs can migrate to secondary lymphoid organs at a greater rate than inactive B cells, or B cells which have not been exposed to Chlamydia spp. (including C. trachomatis, C. psittaci, C. pneumoniae and C. muridarum) or a protein or fragment thereof. For example, the CABs can migrate at a rate of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 90, or 100% faster when compared to a population of cells which have not been exposed to Chlamydia spp. (including C. trachomatis, C. psittaci, C. pneumoniae and C. muridarum) or a protein or fragment thereof. The CABs can migrate at a rate which is 2, 3, 4, 5, 6, 7, 8, 9, 10, 11, 12, 13, 14, 15, 16, 17, 18, 19, 20, or more fold compared to a population of cells which have not been exposed to Chlamydia spp. (including C. trachomatis, C. psittaci, C. pneumoniae and C. muridarum) or a protein or fragment thereof.

The CABs can secrete cytokines to enhance T cell recruitment at a greater rate than inactive B cells. For example, the CABs can recruit T-cell enhancement by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 90, or 100% greater rate when compared to a population of cells which have not been exposed to Chlamydia spp. (including C. trachomatis, C. psittaci, C. pneumoniae and C. muridarum) or a protein or fragment thereof. The CABs enhance T cell recruitment at a rate 2, 3, 4, 5, 6, 7, 8, 9, 10, 11, 12, 13, 14, 15, 16, 17, 18, 19, 20, or more fold compared to a population of cells which have not been exposed to Chlamydia spp. (including C. trachomatis, C. psittaci, C. pneumoniae and C. muridarum) or a protein or fragment thereof.

The CABs disclosed herein can have their activity further enhanced by contacting them with a B cell activating factor, e.g., any of a variety of cytokines, growth factors or cell lines known to activate and/or differentiate B cells (see e.g., Fluckiger, et al. Blood 1998 92: 4509-4520; Luo, et al., Blood 2009 1 13: 1422-1431). Such factors may be selected from the group consisting of, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35, IFN-γ, IFN-α, IFN-β, IFN-δ, C type chemokines XCL1 and XCL2, C-C type chemokines and CXC type chemokines, and members of the TNF superfamily {e.g., TNF-a, 4-1 BB ligand, B cell activating factor (BLyS), FAS ligand, sCD40L (including multimeric versions of sCD40L; e.g., histidine-tagged soluble recombinant CD40L in combination with anti-poly-histidine mAb to group multiple sCD40L molecules together), Lymphotoxin, OX40L, RANKL, TRAIL), CpG, and other Toll like receptor agonists.

In one embodiment, in particular, CABs can be contacted or cultured on feeder cells. In other embodiments, the culture system described herein is carried out in the absence of feeder cells, providing advantages over other systems known in the art that require feeder cells. Where feeder cells may be used, the feeder cells are a stromal cell line, e.g., the murine stromal cell lines S17 or MS5. In a further embodiment, purified CD19⁺ cells may be cultured in the presence of fibroblasts expressing CD40-ligand in the presence of B cell activating cytokines such as IL-10 and IL-4. CD40L may also be provided bound to a surface such as tissue culture plate or a bead. In another embodiment, purified B cells may be cultured in the presence or absence of feeder cells, with CD40L in presence of one or more cytokines or factors selected from IL-10, IL-4, IL-7, p-ODN, CpG DNA, IL-2, IL-15, IL6, IFN-α, and IFN-δ.

In another embodiment, B cell activating factors may be provided by transfection into the B cell or other feeder cell, such as disclosed in PCT/US2000/030426, herein incorporated by reference in its entirety for its teaching concerning CD40L and B cells. In this context, one or more factors that promote differentiation of the B cell into an antibody secreting cell and/or one or more factors that promote the longevity of the antibody producing cell may be used. Such factors include, for example, Blimp-1, TRF4, anti-apoptotic factors like Bcl-xl or Bcl5, or constitutively active mutants of the CD40 receptor. Further, factors which promote expression of downstream signaling molecules such as TNF receptor-associated factors (TRAFs) may also be used in the activation/differentiation of the B cells. In this regard, cell activation, cell survival, and anti-apoptotic functions of the TNF receptor superfamily are mostly mediated by TRAF1-6 (see e.g., R. H. Arch, et al., Genes Dev. 12 (1998), pp. 2821-2830). Downstream effectors of TRAF signaling include transcription factors in the NF-κB and AP-1 family which can turn on genes involved in various aspects of cellular and immune functions. Further, the activation of NF-κB and AP-1 has been shown to provide cells protection from apoptosis via the transcription of anti-apoptotic genes.

The platform can be carried out either ex vivo or in vivo, in whole or in part. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context. For example, the cells can be exposed to an inactivated Chlamydia spp. (including C. trachomatis, C. psittaci, C. pneumoniae and C. muridarum) or peptide or fragment thereof in vivo, or to a live or inactivated Chlamydia spp. (including C. trachomatis, C. psittaci, C. pneumoniae and C. muridarum) or peptide thereof ex vivo, which method is described in more detail herein. The expanded B cells are exposed or crosslinked to an antigen so that they may differentiate accordingly. Again, this can take place in vivo or ex vivo. For example, the antigen may be directly injected into a subject, or B cells of the subject can be exposed or crosslinked to the modified antigen in vitro (ex vivo), with the expanded, differentiated B cells then returned to the subject. The CABs can be used, for example, in direct in vivo administration, ex vivo somatic therapy, in vivo implantable devices and ex vivo extracorporeal devices.

The subject being treated can have a variety of diseases or disorders. Any disease or disorder which can be treated using activated B cells to induce antigen-specific responses in vivo can be treated using the methods disclosed herein. For example, infectious diseases and cancer can be treated using these methods.

Also disclosed is a vaccine comprising the Chlamydia-activated B cells disclosed herein. Disclosed herein is a cell-based vaccine for ex vivo immunization, as well as compositions and methods for in vivo immunization to elicit an immune response directed against an antigen. In one embodiment, disclosed is a subject with a type of cancer which expresses a tumor-specific antigen. This can result in an improved therapeutic outcome for the patient, evidenced by, e.g., a slowing or diminution of the growth of cancer cells or a solid tumor which expresses the tumor-specific antigen, or a reduction in the total number of cancer cells or total tumor burden. In a related embodiment, the patient has been diagnosed as having a viral, bacterial, fungal or other type of infection, which is associated with the expression of a particular antigen, e.g., a viral antigen. This vaccine can result in an improved therapeutic outcome for the patient as evidenced by a slowing in the growth of the causative infectious agent within the patient and/or a decrease in, or elimination of, detectable symptoms typically associated with the particular infectious disease.

When the vaccine is prepared for administration, it can be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” substance is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

The expression vectors, transduced cells, polynucleotides and polypeptides (active ingredients) can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Pharmaceutical formulations containing the therapeutic agents disclosed herein can be prepared by procedures known in the art using well known and readily available ingredients. The therapeutic agents can also be formulated as solutions appropriate for parenteral administration, for instance by intramuscular, subcutaneous or intravenous routes. The pharmaceutical formulations of the therapeutic agents can also take the form of an aqueous or anhydrous solution or dispersion, or alternatively the form of an emulsion or suspension.

The dose given is an amount “effective” in bringing about a desired therapeutic response, be it the stimulation of an immune response, or the treatment of cancer as defined elsewhere in this disclosure. For the pharmaceutical compositions of this invention, effective doses typically fall within the range of about 10⁵ to 10¹¹ cells. Preferably, between about 10⁶ to 10¹⁰ cells are used; more preferably between about 1×10⁷ and 2×10⁹ cells are used. Multiple doses when used in combination to achieve a desired effect each fall within the definition of an effective amount. The doses can be given multiple times a day, or every day, or every other day, or every third day, etc. Additional doses may be given, such as on a monthly or weekly basis, until the desired effect is achieved. Thereafter, and particularly when the immunological or clinical benefit appears to subside, additional booster or maintenance doses may be given as required.

The various components of the cellular vaccine are present in an “effective combination”, which means that there are sufficient amounts of each of the components for the vaccine to be effective. Any number of component cells or other constituents may be used, as long as the vaccine is effective as a whole. This will also depend on the method used to prepare the vaccine.

The pharmaceutical compositions may be given following, preceding, in lieu of, or in combination with, other therapies relating to generating an immune response or treating cancer in the subject. For example, the subject may previously or concurrently be treated by surgical debulking, chemotherapy, radiation therapy, checkpoint inhibitors, and other forms of immunotherapy and adoptive transfer. Where such modalities are used, they are preferably employed in a way or at a time that does not interfere with the immunogenicity of the compositions disclosed herein. The subject may also have been administered another vaccine or other composition in order to stimulate an immune response. Such alternative compositions may include tumor antigen vaccines, nucleic acid vaccines encoding tumor antigens, anti-idiotype vaccines, and other types of cellular vaccines, including cytokine-expressing tumor cell lines.

Disclosed herein are combination therapies, comprising administration of a cellular vaccine combination described herein in conjunction with another strategy aimed at providing an anti-tumor immunological response. In one combination therapy, the subject is given an intra-tumor implant of stimulated allogeneic lymphocytes, either before, during, or after treatment at a site distant from the tumor with a composition comprising the antigen-loaded CABs disclosed herein. In another combination therapy, the subject is treated at sites distant from the tumor with an alternative cellular vaccine composition, either before, during, or after treatment with the antigen-loaded CABs disclosed herein. In another combination therapy, the subject is given checkpoint inhibitors. Where a plurality of different compositions or modes of administration are employed throughout the course of therapy, the order and timing of each element of treatment is chosen to optimize the immunostimulatory or anti-tumor effect of the therapy.

Any of a variety of culture media may be used in the present methods as would be known to the skilled person (see e.g., Current Protocols in Cell Culture, 2000-2009 by John Wiley & Sons, Inc.). In one embodiment, media for use in the methods described herein includes, but is not limited to modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In further embodiments, the medium may comprise a surfactant, an antibody, plasmanate or a reducing agent (e.g. N-acetyl-cysteine, 2-mercaptoethanol), or one or more antibiotics. In some embodiments, IL-2, IL-6, IL-10, soluble CD40L and a cross-linking enhancer may also be used. B cells may be cultured under conditions and for sufficient time periods to achieve activation desired. In certain embodiments, the B cells are cultured under conditions and for sufficient time periods such that 10%, 15%, 20%, 25%, 30%, 35%, 40%, 45%, 50%, 55%, 60%, 65%, 70%, 75%, 80%, 85%, 90%, 95%, or even 100% of the B cells are activated as desired.

In one example, CABs can be generated by using a negative immunomagnetic selection system or RosetteSep™ system to deplete all cell types except CD4⁺ T cells and B cells from PBMC or whole blood. Selected cells are cultured in the presence of Chlamydia spp. and IL-2 (with cell passages every 1-5 days (preferably every 2-3 days) that replenishes media containing Chlamydia spp. and IL-2), until adequate numbers of CABs are produced for use with the methods herein. At the time of harvest for use, flow cytometric evaluation of CD4⁺ T cell frequency can be used to determine if further immunomagnetic selection is needed for further CD4⁺ T cell depletion.

The induction of B cell activation may be measured by techniques such as ³H-thymidine incorporation, which measures DNA synthesis associated with cell proliferation, or by flow cytometric assays using fluorescent markers such as carboxyfluorescein succinimidyl ester. For optimal measurement of B cell proliferation, IL-2 or IL-4 may be added to the culture medium at appropriate concentrations. Alternatively, B cell activation may be measured as a function of immunoglobulin secretion.

After culture for an appropriate period of time, such as 2, 3, 4, 5, 6, 7, 8, 9, or more days (generally around 3 days), an additional volume of culture medium may be added. Supernatant from individual cultures may be harvested at various times during culture and quantitated for IgM and 1gG1 as described in Noelle et al., (1991) J. Immunol. 146:1 1 18-1 124. In further embodiments, enzyme-linked immunosorbent assay (ELISA) may be used for measuring IgM or other antibody isotype. In certain embodiments, IgG determinations may be made using commercially available antibodies such as goat antihuman IgG, as capture antibody, followed by detection using any of a variety of appropriate detection reagents such as biotinylated goat antihuman Ig, streptavidin alkaline phosphatase and substrate.

In one example, different E. coli strains can be used for protein production. For example, CD43(DE3)/pLysS (reference doc. No. 8757792 on PubMed) can be used, and it can induce Ct-MOMP expression with IPTG.

The signal sequence of Ct-MOMP encoded on pET23/42ChOmpA can also be altered to increase translocation across the inner membrane. Strategies include replacement of the current signal sequence (from native E. coli OmpA) for that of another E. coli protein and altering existing sequence through site-specific mutations. Disclosed herein is the use of MOMP or an antigenic fragment thereof with any signal sequence known to those of skill in the art which can increase translocation across the inner membrane.

Also disclosed herein is a method of increasing production of periplasmic chaperones (such as SurA) and folding factors (the Bam complex) in E. coli strains producing Ct-MOMP either by introduction of plasmids or chromosomal mutations that increase their production. These factors are known to those of skill in the art, and are readily available. They can comprise mutations as described in the literature to increase their expression.

Also disclosed are methods to limit competition for folding factors by deleting genes encoding native outer membrane proteins in E. coli strains producing Ct-MOMP. Standard genetic methods can be used.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

SEQUENCES
SEQ ID NO: 1: DNA sequence of native ompA gene encoding MOMP
from Chlamydia trachomatis strain L2/434 (GenBank: DQ064295.1)
Underlined = sequence encoding signal sequence of MOMP
Remainder = sequence encoding mature MOMP
ATGAAAAAACTCTTGAAATCGGTATTAGTGTTTGCCGCTTTGAGTTCTGCTTCCTCCTT
GCAAGCTCTGCCTGTGGGGAATCCTGCTGAACCAAGCCTTATGATCGACGGAATTCTA
TGGGAAGGTTTCGGCGGAGATCCTTGCGATCCTTGCACCACTTGGTGTGACGCTATCA
GCATGCGTATGGGTTACTATGGTGACTTTGTTTTCGACCGTGTTTTGCAAACAGATGTG
AATAAAGAATTCCAAATGGGTGCCAAGCCTACAACTGCTACAGGCAATGCTGCAGCT
CCATCCACTTGTACAGCAAGAGAGAATCCTGCTTACGGCCGACATATGCAGGATGCTG
AGATGTTTACAAATGCTGCTTACATGGCATTGAATATTTGGGATCGTTTTGATGTATTCT
GTACATTAGGAGCCACCAGTGGATATCTTAAAGGAAATTCAGCATCTTTCAACTTAGT
TGGCTTATTCGGAGATAATGAGAACCATGCTACAGTTTCAGATAGTAAGCTTGTACCA
AATATGAGCTTAGATCAATCTGTTGTTGAGTTGTATACAGATACTACTTTTGCTTGGAG
TGCTGGAGCTCGTGCAGCTTTGTGGGAATGTGGATGCGCGACTTTAGGCGCTTCTTTC
CAATACGCTCAATCCAAGCCTAAAGTCGAAGAATTAAACGTTCTCTGTAACGCAGCTG
AGTTTACTATCAATAAGCCTAAAGGATATGTAGGGCAAGAATTCCCTCTTGATCTTAAA
GCAGGAACAGATGGTGTGACAGGAACTAAGGATGCCTCTATTGATTACCATGAATGGC
AAGCAAGTTTAGCTCTCTCTTACAGACTGAATATGTTCACTCCCTACATTGGAGTTAA
ATGGTCTCGAGCAAGTTTTGATGCAGACACGATTCGTATTGCTCAGCCGAAGTCAGCT
ACAACTGTCTTTGATGTTACCACTCTGAACCCAACTATTGCTGGAGCTGGCGATGTGA
AAGCTAGCGCAGAGGGTCAGCTCGGAGATACCATGCAAATCGTTTCCTTGCAATTGA
ACAAGATGAAATCTAGAAAATCTTGCGGTATTGCAGTAGGAACAACTATTGTGGATGC
AGACAAATACGCAGTTACAGTTGAGACTCGCTTGATCGATGAGAGAGCTGCTCACGT
AAATGCACAATTCCGCTTCTAA
SEQ ID NO: 2: Protein sequence of native MOMP from Chlamydia
trachomatis strain L2/434 (GenBank: ABB51013.1)
Underlined = sequence corresponding to signal sequence of MOMP
Remainder = sequence corresponding to mature MOMP
MKKLLKSVLVFAALSSASSLQALPVGNPAEPSLMIDGILWEGFGGDPCDPCTTWCDAISM
RMGYYGDFVFDRVLQTDVNKEFQMGAKPTTATGNAAAPSTCTARENPAYGRHMQDAE
MFTNAAYMALNIWDRFDVFCTLGATSGYLKGNSASFNLVGLFGDNENHATVSDSKLVP
NMSLDQSVVELYTDTTFAWSAGARAALWECGCATLGASFQYAQSKPKVEELNVLCNAA
EFTINKPKGYVGQEFPLDLKAGTDGVTGTKDASIDYHEWQASLALSYRLNMFTPYIGVK
WSRASFDADTIRIAQPKSATTVFDVTTLNPTIAGAGDVKASAEGQLGDTMQIVSLQLNK
MKSRKSCGIAVGTTIVDADKYAVTVETRLIDERAAHVNAQFRF.
SEQ ID NO: 3: DNA sequence of codon-optimized ompA gene
encoding MOMP from Chlamydia trachomatis strain L2/434.
Underlined = sequence encoding signal sequence of MOMP
Remainder = sequence encoding mature MOMP
ATGAAAAAACTGCTGAAATCTGTTCTGGTTTTCGCGGCGCTGTCTTCTGCGTCTTCTC
TGCAGGCGCTGCCGGTTGGTAACCCGGCGGAACCGTCTCTGATGATCGACGGTATCC
TGTGGGAAGGTTTCGGTGGTGACCCGTGCGACCCGTGCACCACCTGGTGCGACGCG
ATCTCTATGCGTATGGGTTACTACGGTGACTTCGTTTTCGACCGTGTTCTGCAGACCGA
CGTTAACAAAGAATTCCAGATGGGTGCGAAACCGACCACCGCGACCGGTAACGCGG
CGGCGCCGTCTACCTGCACCGCGCGTGAAAACCCGGCGTACGGTCGTCACATGCAGG
ACGCGGAAATGTTCACCAACGCGGCGTACATGGCGCTGAACATCTGGGACCGTTTCG
ACGTTTTCTGCACCCTGGGTGCGACCTCTGGTTACCTGAAAGGTAACTCTGCGTCTTT
CAACCTGGTTGGTCTGTTCGGTGACAACGAAAACCACGCGACCGTTTCTGACTCTAA
ACTGGTTCCGAACATGTCTCTGGACCAGTCTGTTGTTGAACTGTACACCGACACCAC
CTTCGCGTGGTCTGCGGGTGCGCGTGCGGCGCTGTGGGAATGCGGTTGCGCGACCCT
GGGTGCGTCTTTCCAGTACGCGCAGTCTAAACCGAAAGTTGAAGAACTGAACGTTCT
GTGCAACGCGGCGGAATTCACCATCAACAAACCGAAAGGTTACGTTGGTCAGGAATT
CCCGCTGGACCTGAAAGCGGGTACCGACGGTGTTACCGGTACCAAAGACGCGTCTAT
CGACTACCACGAATGGCAGGCGTCTCTGGCGCTGTCTTACCGTCTGAACATGTTCACC
CCGTACATCGGTGTTAAATGGTCTCGTGCGTCTTTCGACGCGGACACCATCCGTATCG
CGCAGCCGAAATCTGCGACCACCGTTTTCGACGTTACCACCCTGAACCCGACCATCG
CGGGTGCGGGTGACGTTAAAGCGTCTGCGGAAGGTCAGCTGGGTGACACCATGCAG
ATCGTTTCTCTGCAGCTGAACAAAATGAAATCTCGTAAATCTTGCGGTATCGCGGTTG
GTACCACCATCGTTGACGCGGACAAATACGCGGTTACCGTTGAAACCCGTCTGATCG
ACGAACGTGCGGCGCACGTTAACGCGCAGTTCCGTTTCTAA
SEQ ID NO: 4: DNA sequence of codon-optimized ompA gene
encoding mature MOMP from Chlamydia trachomatis strain L2/434.
CTGCCGGTTGGTAACCCGGCGGAACCGTCTCTGATGATCGACGGTATCCTGTGGGAA
GGTTTCGGTGGTGACCCGTGCGACCCGTGCACCACCTGGTGCGACGCGATCTCTATG
CGTATGGGTTACTACGGTGACTTCGTTTTCGACCGTGTTCTGCAGACCGACGTTAACA
AAGAATTCCAGATGGGTGCGAAACCGACCACCGCGACCGGTAACGCGGCGGCGCCG
TCTACCTGCACCGCGCGTGAAAACCCGGCGTACGGTCGTCACATGCAGGACGCGGAA
ATGTTCACCAACGCGGCGTACATGGCGCTGAACATCTGGGACCGTTTCGACGTTTTCT
GCACCCTGGGTGCGACCTCTGGTTACCTGAAAGGTAACTCTGCGTCTTTCAACCTGG
TTGGTCTGTTCGGTGACAACGAAAACCACGCGACCGTTTCTGACTCTAAACTGGTTC
CGAACATGTCTCTGGACCAGTCTGTTGTTGAACTGTACACCGACACCACCTTCGCGT
GGTCTGCGGGTGCGCGTGCGGCGCTGTGGGAATGCGGTTGCGCGACCCTGGGTGCGT
CTTTCCAGTACGCGCAGTCTAAACCGAAAGTTGAAGAACTGAACGTTCTGTGCAACG
CGGCGGAATTCACCATCAACAAACCGAAAGGTTACGTTGGTCAGGAATTCCCGCTGG
ACCTGAAAGCGGGTACCGACGGTGTTACCGGTACCAAAGACGCGTCTATCGACTACC
ACGAATGGCAGGCGTCTCTGGCGCTGTCTTACCGTCTGAACATGTTCACCCCGTACAT
CGGTGTTAAATGGTCTCGTGCGTCTTTCGACGCGGACACCATCCGTATCGCGCAGCCG
AAATCTGCGACCACCGTTTTCGACGTTACCACCCTGAACCCGACCATCGCGGGTGCG
GGTGACGTTAAAGCGTCTGCGGAAGGTCAGCTGGGTGACACCATGCAGATCGTTTCT
CTGCAGCTGAACAAAATGAAATCTCGTAAATCTTGCGGTATCGCGGTTGGTACCACCA
TCGTTGACGCGGACAAATACGCGGTTACCGTTGAAACCCGTCTGATCGACGAACGTG
CGGCGCACGTTAACGCGCAGTTCCGTTTCTAA
SEQ ID NO: 5: DNA sequence from the Escherichia coli ompA gene
encoding the signal sequence of the outer membrane protein OmpA.
Sequence was derived from ompA gene, locus tag b0957 in genome
from E. coli str. K-12 substr. MG1655 (GenBank: U00096.3)
ATGAAAAAGACAGCTATCGCGATTGCAGTGGCACTGGCTGGTTTCGCTACCGTAGCG
CAGGCC
SEQ ID NO: 6: Protein sequence corresponding to the signal
sequence of the Escherichia coli outer membrane protein
OmpA (from GenBank: AAC74043.1).
MKKTAIAIAVALAGFATVAQA
SEQ ID NO: 7: DNA sequence of the hybrid gene (ompA_{Ec-Ct})
encoding the signal sequence of E. coli OmpA fused to the
C. trachomatis mature MOMP.
The DNA sequence encoding the signal sequence from E. coli
OmpA (ompA gene) is underlined.
The DNA sequence encoding the mature portion of MOMP is derived
from the codon-optimized allele, and is not underlined.
ATGAAAAAGACAGCTATCGCGATTGCAGTGGCACTGGCTGGTTTCGCTACCGTAGCG
CAGGCCCTGCCGGTTGGTAACCCGGCGGAACCGTCTCTGATGATCGACGGTATCCTGT
GGGAAGGTTTCGGTGGTGACCCGTGCGACCCGTGCACCACCTGGTGCGACGCGATCT
CTATGCGTATGGGTTACTACGGTGACTTCGTTTTCGACCGTGTTCTGCAGACCGACGT
TAACAAAGAATTCCAGATGGGTGCGAAACCGACCACCGCGACCGGTAACGCGGCGG
CGCCGTCTACCTGCACCGCGCGTGAAAACCCGGCGTACGGTCGTCACATGCAGGACG
CGGAAATGTTCACCAACGCGGCGTACATGGCGCTGAACATCTGGGACCGTTTCGACG
TTTTCTGCACCCTGGGTGCGACCTCTGGTTACCTGAAAGGTAACTCTGCGTCTTTCAA
CCTGGTTGGTCTGTTCGGTGACAACGAAAACCACGCGACCGTTTCTGACTCTAAACT
GGTTCCGAACATGTCTCTGGACCAGTCTGTTGTTGAACTGTACACCGACACCACCTTC
GCGTGGTCTGCGGGTGCGCGTGCGGCGCTGTGGGAATGCGGTTGCGCGACCCTGGGT
GCGTCTTTCCAGTACGCGCAGTCTAAACCGAAAGTTGAAGAACTGAACGTTCTGTGC
AACGCGGCGGAATTCACCATCAACAAACCGAAAGGTTACGTTGGTCAGGAATTCCCG
CTGGACCTGAAAGCGGGTACCGACGGTGTTACCGGTACCAAAGACGCGTCTATCGAC
TACCACGAATGGCAGGCGTCTCTGGCGCTGTCTTACCGTCTGAACATGTTCACCCCGT
ACATCGGTGTTAAATGGTCTCGTGCGTCTTTCGACGCGGACACCATCCGTATCGCGCA
GCCGAAATCTGCGACCACCGTTTTCGACGTTACCACCCTGAACCCGACCATCGCGGG
TGCGGGTGACGTTAAAGCGTCTGCGGAAGGTCAGCTGGGTGACACCATGCAGATCGT
TTCTCTGCAGCTGAACAAAATGAAATCTCGTAAATCTTGCGGTATCGCGGTTGGTACC
ACCATCGTTGACGCGGACAAATACGCGGTTACCGTTGAAACCCGTCTGATCGACGAA
CGTGCGGCGCACGTTAACGCGCAGTTCCGTTTCTAA
SEQ ID NO: 8: Protein sequence of the hybrid protein
OmpA_{Ec}-MOMP_{Ct} encoding the signal sequence of E. coli OmpA
fused to the C. trachomatis mature MOMP.
(Signal sequence is underlined, remainder is mature MOMP).
MKKTAIAIAVALAGFATVAQALPVGNPAEPSLMIDGILWEGFGGDPCDPCTTWCDAISMR
MGYYGDFVFDRVLQTDVNKEFQMGAKPTTATGNAAAPSTCTARENPAYGRHMQDAEM
FTNAAYMALNIWDRFDVFCTLGATSGYLKGNSASFNLVGLFGDNENHATVSDSKLVPN
MSLDQSVVELYTDTTFAWSAGARAALWECGCATLGASFQYAQSKPKVEELNVLCNAAE
FTINKPKGYVGQEFPLDLKAGTDGVTGTKDASIDYHEWQASLALSYRLNMFTPYIGVK
WSRASFDADTIRIAQPKSATTVFDVTTLNPTIAGAGDVKASAEGQLGDTMQIVSLQLNK
MKSRKSCGIAVGTTIVDADKYAVTVETRLIDERAAHVNAQFRF.
SEQ ID NO: 9: Sequence of the hybrid gene (ompAΔcys_{Ec-Ct}) encoding
the signal sequence of E. coli OmpA fused to the C. trachomatis
mature Cys-less MOMP (signal sequence is underlined, remainder
is mature Cys-less MOMP).
Sequence from SEQ ID NO: 7 was altered to substitute the
9 Cys codons (TGC) for Ser codons (TCT).
ATGAAAAAGACAGCTATCGCGATTGCAGTGGCACTGGCTGGTTTCGCTACCGTAGCG
CAGGCCCTGCCGGTTGGTAACCCGGCGGAACCGTCTCTGATGATCGACGGTATCCTGT
GGGAAGGTTTCGGTGGTGACCCGTCTGACCCGTCTACCACCTGGTCTGACGCGATCT
CTATGCGTATGGGTTACTACGGTGACTTCGTTTTCGACCGTGTTCTGCAGACCGACGT
TAACAAAGAATTCCAGATGGGTGCGAAACCGACCACCGCGACCGGTAACGCGGCGG
CGCCGTCTACCTCTACCGCGCGTGAAAACCCGGCGTACGGTCGTCACATGCAGGACG
CGGAAATGTTCACCAACGCGGCGTACATGGCGCTGAACATCTGGGACCGTTTCGACG
TTTTCTCTACCCTGGGTGCGACCTCTGGTTACCTGAAAGGTAACTCTGCGTCTTTCAA
CCTGGTTGGTCTGTTCGGTGACAACGAAAACCACGCGACCGTTTCTGACTCTAAACT
GGTTCCGAACATGTCTCTGGACCAGTCTGTTGTTGAACTGTACACCGACACCACCTTC
GCGTGGTCTGCGGGTGCGCGTGCGGCGCTGTGGGAATCTGGTTCTGCGACCCTGGGT
GCGTCTTTCCAGTACGCGCAGTCTAAACCGAAAGTTGAAGAACTGAACGTTCTGTCT
AACGCGGCGGAATTCACCATCAACAAACCGAAAGGTTACGTTGGTCAGGAATTCCCG
CTGGACCTGAAAGCGGGTACCGACGGTGTTACCGGTACCAAAGACGCGTCTATCGAC
TACCACGAATGGCAGGCGTCTCTGGCGCTGTCTTACCGTCTGAACATGTTCACCCCGT
ACATCGGTGTTAAATGGTCTCGTGCGTCTTTCGACGCGGACACCATCCGTATCGCGCA
GCCGAAATCTGCGACCACCGTTTTCGACGTTACCACCCTGAACCCGACCATCGCGGG
TGCGGGTGACGTTAAAGCGTCTGCGGAAGGTCAGCTGGGTGACACCATGCAGATCGT
TTCTCTGCAGCTGAACAAAATGAAATCTCGTAAATCTTCTGGTATCGCGGTTGGTACC
ACCATCGTTGACGCGGACAAATACGCGGTTACCGTTGAAACCCGTCTGATCGACGAA
CGTGCGGCGCACGTTAACGCGCAGTTCCGTTTCTAA
SEQ ID NO: 10: Sequence of the hybrid protein gene
OmpA_{Ec}-MOMPΔCys_{Ct} (ompAΔcys_{Ec-Ct}) encoding the signal sequence
of E. coli OmpA fused to the C. trachomatis mature Cys-less
MOMP (underlined = signal sequence, remainder = Cys-less MOMP).
MKKTAIAIAVALAGFATVAQALPVGNPAEPSLMIDGILWEGFGGDPSDPSTTWSDAISMR
MGYYGDFVFDRVLQTDVNKEFQMGAKPTTATGNAAAPSTSTARENPAYGRHMQDAEM
FTNAAYMALNIWDRFDVFSTLGATSGYLKGNSASFNLVGLFGDNENHATVSDSKLVPN
MSLDQSVVELYTDTTFAWSAGARAALWESGSATLGASFQYAQSKPKVEELNVLSNAAEF
TINKPKGYVGQEFPLDLKAGTDGVTGTKDASIDYHEWQASLALSYRLNMFTPYIGVKW
SRASFDADTIRIAQPKSATTVFDVTTLNPTIAGAGDVKASAEGQLGDTMQIVSLQLNKM
KSRKSSGIAVGTTIVDADKYAVTVETRLIDERAAHVNAQFRF.
SEQ ID NO: 11: C. trachomatis mature Cys-less MOMP
LPVGNPAEPSLMIDGILWEGFGGDPSDPSTTWSDAISMRMGYYGDFVFDRVLQTDVNKE
FQMGAKPTTATGNAAAPSTSTARENPAYGRHMQDAEMFTNAAYMALNIWDRFDVFSTL
GATSGYLKGNSASFNLVGLFGDNENHATVSDSKLVPNMSLDQSVVELYTDTTFAWSAGA
RAALWESGSATLGASFQYAQSKPKVEELNVLSNAAEFTINKPKGYVGQEFPLDLKAGTD
GVTGTKDASIDYHEWQASLALSYRLNMFTPYIGVKWSRASFDADTIRIAQPKSATTVFD
VTTLNPTIAGAGDVKASAEGQLGDTMQIVSLQLNKMKSRKSSGIAVGTTIVDADKYAVT
VETRLIDERAAHVNAQFRF

1-8. (canceled)

9. A platform for creating activated, antigen-presenting cells (APCs), wherein the platform comprises:
a. a polypeptide or glycopolypeptide comprising a derivative of naturally occurring major outer membrane protein (MOMP) of a Chlamydia spp. or an antigenic fragment thereof, wherein the naturally occurring MOMP is modified so that at least one cysteine residue of MOMP is replaced with a different amino acid;
b. a population of B cells; and
c. an antigen, wherein the antigen is not derived from a Chlamydia spp.

10. The platform of claim 9, wherein the polypeptide comprises a signal sequence which is not naturally occurring with Chlamydial-derived MOM.

11. The platform of claim 10, wherein the signal sequence is derived from Escherichia coli.

12. The platform of claim 11, wherein the signal sequence is at least 90% identical to an amino acid sequence comprising SEQ ID NO: 6.

13. The platform of claim 9, wherein MOMP is derived from C. trachomatis, C. psittaci, C. pneumoniae or C. muridarum, or another Chlamydia spp., and modified to substitute at least one cysteine residue with a different amino acid.

14. The platform of claim 9, wherein the at least one cysteine residue has been replaced with a serine residue.

15. The platform of claim 9, wherein antigenic fragment of MOMP comprises one or more surface-exposed fragments of MOMP.

16. A vaccine created using the platform of claim 9.

17. A Chlamydia-activated B cell produced by the platform of claim 9.

18. A method for producing activated, antigen-presenting Chlamydia-activated B cells in a subject, the method comprising:
a. transforming a cell with a plasmid, wherein the plasmid comprises a nucleic acid encoding a derivative of major outer membrane (MOMP) protein or an antigenic fragment thereof, and a signal sequence, thereby producing recombinant MOMP;
b. exposing B cells of the subject to the recombinant MOMP of step a);
c. exposing the B cells of step b) to a desired antigen, wherein the antigen is not derived from Chlamydia spp., thereby obtaining activated, antigen-presenting Chlamydia-activated B cells (CABs).

19. The method of claim 18, wherein the cell transformed with a plasmid is Escherichia coli.

20. The method of claim 19, wherein the signal sequence is E. coli OmpA.

21. The method of claim 20, wherein the signal sequence comprises SEQ ID NO: 5.

22. The method of claim 18, wherein MOMP is derived from C. trachomatis, C. psittaci, C. pneumoniae or C. muridarum, or another Chlamydia spp., and modified to substitute at least one cysteine with a different amino acid.

23. The method of claim 18, wherein all cysteine residues of a naturally occurring MOMP are replaced with serine residues.

24. The method of claim 23, wherein after exposing the B cells from step a) to a polypeptide comprising MOMP, wherein the naturally occurring MOMP is modified so that at least one cysteine residue of MOMP is replaced with a different amino acid; adding the step of crosslinking a protein, peptide, nucleic acid, lipid, carbohydrate or fragment thereof to the CABs, wherein the protein, peptide, nucleic acid, lipid, carbohydrate or fragment thereof is antigenic.

25. The method of claim 18 wherein the antigenic fragment of MOMP comprises one or more surface exposed fragments of MOMP.

26. A Chlamydia-activated B cell produced by the method of claim 18.

27-32. (canceled)

33. A vaccine comprising an antigen and a polypeptide comprising a derivative of naturally occurring major outer membrane protein (MOMP) of a Chlamydia spp. or an antigenic fragment thereof, wherein the naturally occurring MOMP is modified so that at least one cysteine residue of MOMP is replaced with a different amino acid, and wherein the antigen is not derived from a Chlamydia spp.

34.-41. (canceled)

42. The vaccine of claim 33, wherein the vaccine increases specific humoral immunity (e.g., antibody affinity maturation or strength of antibody binding to cognate antigen) as compared to the humoral immune response induced by antigen alone.

43-47. (canceled)
